(19) United States
(12) Patent Application Publication
(10) Pub. No.: US 2019/0087544 A1
(43) Pub. Date: Mar. 21, 2019

(54) Surgery Digital Twin

(71) Applicant: General Electric Company, Schenectady, NY (US)

(72) Inventor: Marcia Peterson, Barrington, IL (US)

(21) Appl. No.: 15/711,786

(22) Filed: Sep. 21, 2017

(51) Int. Cl. G06F 19/00; G06Q 50/24

(52) U.S. Cl. CPC G06F 19/3437; G06F 19/345; A61B 2034/105; G06F 19/322; G06F 19/3418; G06Q 50/24

(57) ABSTRACT

Methods and apparatus providing a digital twin are disclosed. An example apparatus includes a digital twin of a healthcare procedure. The example digital twin includes a data structure created from tasks defining the healthcare procedure and items to be used in the healthcare procedure to model the tasks and items associated with each task for query and simulation for a patient. The example digital twin is to at least: receive input regarding a first item at a location; compare the first item to the items associated with each task; and, when the first item matches an item associated with a task of the healthcare procedure, record the first item and approval for the healthcare procedure and update the digital twin based on the first item. When the first item does not match an item associated with a task, the example digital twin is to log the first item.

### FIELD OF THE DISCLOSURE

This disclosure relates generally to improved patient and healthcare operation modeling and care and, more particularly, to improved systems and methods for improving patient care through surgical tracking, feedback, and analysis, such as using a digital twin.

### BACKGROUND

A variety of economic, technological, and administrative hurdles challenge healthcare facilities, such as hospitals, clinics, doctors' offices, etc., to provide quality care to patients. Economic drivers, evolving medical science, less and skilled staff, fewer staff, complicated equipment, and emerging accreditation for controlling and standardizing radiation exposure dose usage across a healthcare enterprise create difficulties for effective management and use of imaging and information systems for examination, diagnosis, and treatment of patients.

Healthcare provider consolidations create geographically distributed hospital networks in which physical contact with systems is too costly. At the same time, referring physicians want more direct access to supporting data in reports and other data forms along with better channels for collaboration. Physicians have more patients, less time, and are inundated with huge amounts of data, and they are eager for assistance.

### BRIEF SUMMARY

Certain examples provide an apparatus including a processor and a memory. The example processor is to configure the memory according to a digital twin of a first healthcare procedure. The example digital twin includes a data structure created from tasks defining the first healthcare procedure and a list of items to be used in the first healthcare procedure to model the tasks of the first healthcare procedure and items associated with each task of the first healthcare procedure. The example digital twin is arranged for query and simulation via the processor to model the first healthcare procedure for a first patient. The example digital twin is to at least: receive input regarding a first item at a first location; compare the first item to the items associated with each task of the first healthcare procedure; and, when the first item matches an item associated with a task of the first healthcare procedure, record the first item and approval for the first healthcare procedure and update the digital twin based on the first item. When the first item does not match an item associated with a task of the first healthcare procedure, the example digital twin is to log the first item.

Certain examples provide a computer-readable storage medium including instructions which, when executed by a processor, cause a machine to implement at least a digital twin of a first healthcare procedure. The example digital twin includes a data structure created from tasks defining the first healthcare procedure and a list of items to be used in the first healthcare procedure to model the tasks of the first healthcare procedure and items associated with each task of the first healthcare procedure. The example digital twin is arranged for query and simulation via the processor to model the first healthcare procedure for a first patient. The example digital twin is to at least: receive input regarding a first item at a first location; compare the first item to the items associated with each task of the first healthcare procedure; and, when the first item matches an item associated with a task of the first healthcare procedure, record the first item and approval for the first healthcare procedure and update the digital twin based on the first item. When the first item does not match an item associated with a task of the first healthcare procedure, the example digital twin is to log the first item.

Certain examples provide a method including receiving, using a processor, input regarding a first item at a first location. The example method includes comparing, using the processor, the first item to items associated with each task of a first healthcare procedure, the items associated with each task of the first healthcare protocol modeled using a digital twin of the first healthcare protocol, the digital twin including a data structure created from tasks defining the first healthcare procedure and a list of items to be used in the first healthcare procedure to model the tasks of the first healthcare procedure and items associated with each task of the first healthcare procedure, the digital twin arranged for query and simulation via the processor to model the first healthcare procedure for a first patient. The example method includes, when the first item matches an item associated with a task of the first healthcare procedure, recording the first item and approval for the first healthcare procedure and update the digital twin based on the first item. The example method includes, when the first item does not match an item associated with a task of the first healthcare procedure, logging the first item.

### BRIEF DESCRIPTION OF THE DRAWINGS

FIG. 1 illustrates a patient/procedure in a real space providing data to a digital twin in a virtual space.

FIG. 2 illustrates an example implementation of a surgery digital twin.

FIG. 3 shows an example optical head-mounted display including a scanner to scan items in its field of view.

FIG. 4 shows an example instrument cart including a computing device operating with respect to a digital twin.

FIG. 5 illustrates an example monitored environment for a digital twin.

FIG. 6 illustrates an example instrument processing facility for processing/re-processing instruments.

FIG. 7 illustrates an example operating room monitor including a digital twin.

FIG. 8 illustrates an example ecosystem to facilitate trending and tracking of surgical procedures and other protocol compliance via a digital twin.

FIG. 9 illustrates a flow diagram of an example process for procedure modeling using a digital twin.

FIG. 10 presents an example augmented reality visualization including auxiliary information regarding various aspects of an operating room environment.

FIG. 11 provides further detail regarding updating of the digital twin of the method of FIG. 9.

FIG. 12 illustrates an example preference card for an arthroscopic orthopedic procedure modeled using a digital twin.

FIG. 13 provides further detail regarding monitoring procedure execution of the method of FIG. 9.

FIG. 14 is a representation of an example deep learning neural network that can be used to implement the surgery digital twin.

FIG. 15 shows a block diagram of an example healthcare-focused information system.

FIG. 16 shows a block diagram of an example healthcare information infrastructure.

FIG. 17 illustrates an example industrial internet configuration.

FIG. 18 is a block diagram of a processor platform structured to execute the example machine readable instructions to implement components disclosed and described herein.

The figures are not scale. Wherever possible, the same reference numbers will be used throughout the drawings and accompanying written description to refer to the same or like parts.

### DETAILED DESCRIPTION

In the following detailed description, reference is made to the accompanying drawings that form a part hereof, and in which is shown by way of illustration specific examples that may be practiced. These examples are described in sufficient detail to enable one skilled in the art to practice the subject matter, and it is to be understood that other examples may be utilized and that logical, mechanical, electrical and other changes may be made without departing from the scope of the subject matter of this disclosure. The following detailed description is, therefore, provided to describe an exemplary implementation and not to be taken as limiting on the scope of the subject matter described in this disclosure. Certain features from different aspects of the following description may be combined to form yet new aspects of the subject matter discussed below.

When introducing elements of various embodiments of the present disclosure, the articles “a,” “an,” “the,” and “said” are intended to mean that there are one or more of the elements. The terms “comprising,” “including,” and “having” are intended to be inclusive and mean that there may be additional elements other than the listed elements.

As used herein, the terms “system,” “unit,” “module,” “engine,” etc., may include a hardware and/or software system that operates to perform one or more functions. For example, a module, unit, or system may include a computer processor, controller, and/or other logic-based device that performs operations based on instructions stored on a tangible and non-transitory computer readable storage medium, such as a computer memory. Alternatively, a module, unit, engine, or system may include a hard-wired device that performs operations based on hard-wired logic of the device. Various modules, units, engines, and/or systems shown in the attached figures may represent the hardware that operates based on software or hardwired instructions, the software that directs hardware to perform the operations, or a combination thereof.

While certain examples are described below in the context of medical or healthcare systems, other examples can be implemented outside the medical environment. For example, certain examples can be applied to non-medical imaging such as non-destructive testing, explosive detection, etc.

### I. Overview

A digital representation, digital model, digital “twin”, or digital “shadow” is a digital informational construct about a physical system, process, etc. That is, digital information can be implemented as a “twin” of a physical device/system/person/process and information associated with and/or embedded within the physical device/system/process. The digital twin is linked with the physical system through the lifecycle of the physical system. In certain examples, the digital twin includes a physical object in real space, a digital twin of that physical object that exists in a virtual space, and information linking the physical object with its digital twin. The digital twin exists in a virtual space corresponding to a real space and includes a link for data flow from real space to virtual space as well as a link for information flow from virtual space to real space and virtual sub-spaces.

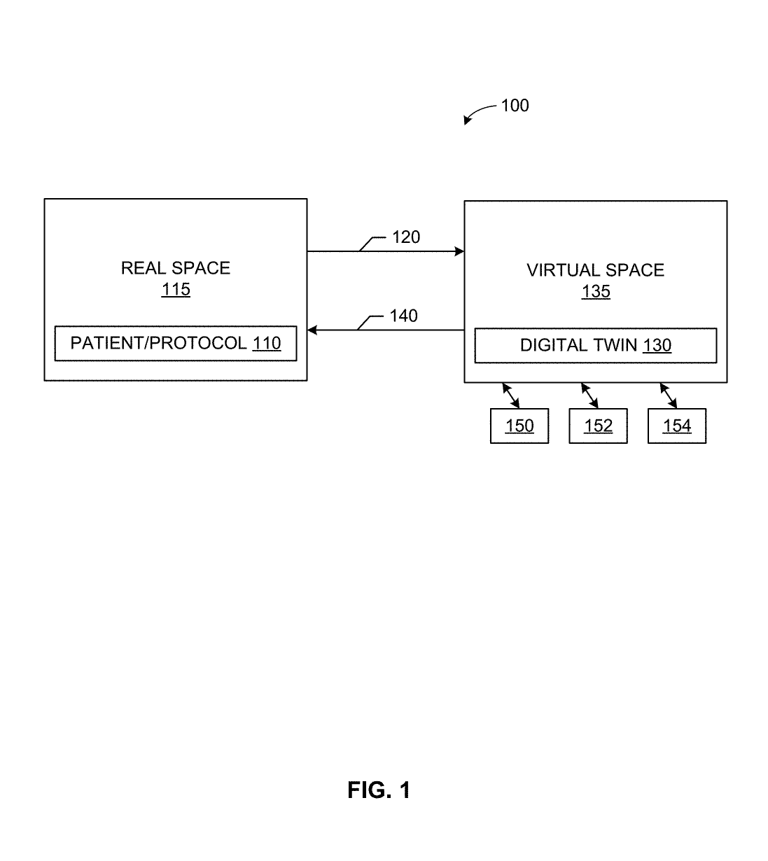

For example, FIG. 1 illustrates a patient, protocol, and/or other item 110 in a real space 115 providing data 120 to a digital twin 130 in a virtual space 135. The digital twin 130 and/or its virtual space 135 provide information 140 back to the real space 115. The digital twin 130 and/or virtual space 135 can also provide information to one or more virtual sub-spaces 150, 152, 154. As shown in the example of FIG. 1, the virtual space 135 can include and/or be associated with one or more virtual sub-spaces 150, 152, 154, which can be used to model one or more parts of the digital twin 130 and/or digital “sub-twins” modeling subsystems/subparts of the overall digital twin 130.

Sensors connected to the physical object (e.g., the patient 110) can collect data and relay the collected data 120 to the digital twin 130 (e.g., via self-reporting, using a clinical or other health information system such as a picture archiving and communication system (PACS), radiology information system (RIS), electronic medical record system (EMR), laboratory information system (LIS), cardiovascular information system (CVIS), hospital information system (HIS), and/or combination thereof, etc.). Interaction between the digital twin 130 and the patient/protocol 110 can help improve diagnosis, treatment, health maintenance, etc., for the patient 110 (such as adherence to the protocol, etc.), for example. An accurate digital description 130 of the patient/protocol/item 110 benefiting from a real-time or substantially real-time (e.g., accounting from data transmission, processing, and/or storage delay) allows the system 100 to predict “failures” in the form of disease, body function, and/or other malady, condition, etc.

In certain examples, obtained images overlaid with sensor data, lab results, etc., can be used in augmented reality (AR) applications when a healthcare practitioner is examining, treating, and/or otherwise caring for the patent 110. Using AR, the digital twin 130 follows the patient's response to the interaction with the healthcare practitioner, for example.

Thus, rather than a generic model, the digital twin 130 is a collection of actual physics-based, anatomically-based, and/or biologically-based models reflecting the patient/protocol/item 110 and his or her associated norms, conditions, etc. In certain examples, three-dimensional (3D) modeling of the patient/protocol/item 110 creates the digital twin 130 for the patient/protocol/item 110. The digital twin 130 can be used to view a status of the patient/protocol/item 110 based on input data 120 dynamically provided from a source (e.g., from the patient 110, practitioner, health information system, sensor, etc.).

In certain examples, the digital twin 130 of the patient/protocol/item 110 can be used for monitoring, diagnostics, and prognostics for the patient/protocol/item 110. Using sensor data in combination with historical information, current and/or potential future conditions of the patient/protocol/item 110 can be identified, predicted, monitored, etc., using the digital twin 130. Causation, escalation, improvement, etc., can be monitored via the digital twin 130. Using the digital twin 130, the patient/protocol/item's 110 physical behaviors can be simulated and visualized for diagnosis, treatment, monitoring, maintenance, etc.

In contrast to computers, humans do not process information in a sequential, step-by-step process. Instead, people try to conceptualize a problem and understand its context. While a person can review data in reports, tables, etc., the person is most effective when visually reviewing a problem and trying to find its solution. Typically, however, when a person visually processes information, records the information in alphanumeric form, and then tries to re-conceptualize the information visually, information is lost and the problem-solving process is made much less efficient over time.

Using the digital twin 130, however, allows a person and/or system to view and evaluate a visualization of a situation (e.g., a patient/protocol/item 110 and associated patient problem, etc.) without translating to data and back. With the digital twin 130 in common perspective with the actual patient/protocol/item 110, physical and virtual information can be viewed together, dynamically and in real time (or substantially real time accounting for data processing, transmission, and/or storage delay). Rather than reading a report, a healthcare practitioner can view and simulate with the digital twin 130 to evaluate a condition, progression, possible treatment, etc., for the patient/protocol/item 110. In certain examples, features, conditions, trends, indicators, traits, etc., can be tagged and/or otherwise labeled in the digital twin 130 to allow the practitioner to quickly and easily view designated parameters, values, trends, alerts, etc.

The digital twin 130 can also be used for comparison (e.g., to the patient/protocol/item 110, to a “normal”, standard, or reference patient, set of clinical criteria/symptoms, best practices, protocol steps, etc.). In certain examples, the digital twin 130 of the patient/protocol/item 110 can be used to measure and visualize an ideal or “gold standard” value state for that patient/protocol/item, a margin for error or standard deviation around that value (e.g., positive and/or negative deviation from the gold standard value, etc.), an actual value, a trend of actual values, etc. A difference between the actual value or trend of actual values and the gold standard (e.g., that falls outside the acceptable deviation) can be visualized as an alphanumeric value, a color indication, a pattern, etc.

Further, the digital twin 130 of the patient 110 can facilitate collaboration among friends, family, care providers, etc., for the patient 110. Using the digital twin 130, conceptualization of the patient 110 and his/her health can be shared (e.g., according to a care plan, etc.) among multiple people including care providers, family, friends, etc. People do not need to be in the same location as the patient 110, with each other, etc., and can still view, interact with, and draw conclusions from the same digital twin 130, for example.

Thus, the digital twin 130 can be defined as a set of virtual information constructs that describes (e.g., fully describes) the patient 110 from a micro level (e.g., heart, lungs, foot, anterior cruciate ligament (ACL), stroke history, etc.) to a macro level (e.g., whole anatomy, holistic view, skeletal system, nervous system, vascular system, etc.). Similarly, the digital twin 130 can represent an item and/or a protocol at various levels of detail such as macro, micro, etc. In certain examples, the digital twin 130 can be a reference digital twin (e.g., a digital twin prototype, etc.) and/or a digital twin instance. The reference digital twin represents a prototypical or “gold standard” model of the patient/protocol/item 110 or of a particular type/category of patient/protocol/item 110, while one or more reference digital twins represent particular patient(s)/protocol(s)/item(s) 110. Thus, the digital twin 130 of a child patient 110 may be implemented as a child reference digital twin organized according to certain standard or “typical” child characteristics, with a particular digital twin instance representing the particular child patient 110. In certain examples, multiple digital twin instances can be aggregated into a digital twin aggregate (e.g., to represent an accumulation or combination of multiple child patients sharing a common reference digital twin, etc.). The digital twin aggregate can be used to identify differences, similarities, trends, etc., between children represented by the child digital twin instances, for example.

In certain examples, the virtual space 135 in which the digital twin 130 (and/or multiple digital twin instances, etc.) operates is referred to as a digital twin environment. The digital twin environment 135 provides an integrated, multi-domain physics- and/or biologics-based application space in which to operate the digital twin 130. The digital twin 130 can be analyzed in the digital twin environment 135 to predict future behavior, condition, progression, etc., of the patient/protocol/item 110, for example. The digital twin 130 can also be interrogated or queried in the digital twin environment 135 to retrieve and/or analyze current information 140, past history, etc.

In certain examples, the digital twin environment 135 can be divided into multiple virtual spaces 150-154. Each virtual space 150-154 can model a different digital twin instance and/or component of the digital twin 130 and/or each virtual space 150-154 can be used to perform a different analysis, simulation, etc., of the same digital twin 130. Using the multiple virtual spaces 150-154, the digital twin 130 can be tested inexpensively and efficiently in a plurality of ways while preserving patient 110 safety. A healthcare provider can then understand how the patient/protocol/item 110 may react to a variety of treatments in a variety of scenarios, for example.

In certain examples, instead of or in addition to the patient/protocol/item 110, the digital twin 130 can be used to model a robot, such as a robot to assist in healthcare monitoring, patient care, care plan execution, surgery, patient follow-up, etc. As with the patient/protocol/item 110, the digital twin 130 can be used to model behavior, programming, usage, etc., for a healthcare robot, for example. The robot can be a home healthcare robot to assist in patient monitoring and in-home patient care, for example. The robot can be programmed for a particular patient condition, care plan, protocol, etc., and the digital twin 130 can model execution of such a plan/protocol, simulate impact on the patient condition, predict next step(s) in patient care, suggest next action(s) to facilitate patient compliance, etc.

In certain examples, the digital twin 130 can also model a space, such as an operating room, surgical center, pre-operative preparation room, post-operative recovery room, etc. By modeling an environment, such as a surgical suite, the environment can be made, safer, more reliable, and/or more productive for patients, healthcare professionals (e.g., surgeons, nurses, anesthesiologists, technicians, etc.). For example, the digital twin 130 can be used for improved instrument and/or surgical item tracking/management, etc.

In certain examples, a cart, table, and/or other set of surgical tools/instructions is brought into an operating room in preparation for surgery. Items on the cart can be inventoried, validated, and modeled using the digital twin 130, for example. For example, items on a surgical cart are validated, and items to be used in a surgical procedure are accounted for (e.g., a list of items to be used in the surgical procedure (e.g., knee replacement, ligament reconstruction, organ removal, etc.) is compared to items on the cart, etc.). Unused items can be returned to stock (e.g., so the patient is not charged for unused/unnecessary items, so incorrect items are not inadvertently used in the procedure, etc.). Items can include one or more surgical implements, wound care items, medications, implants, etc.

Rather than using paper barcodes, nurse inspections, code scanners, etc., which take time and attention away from the patient and lead to inaccuracies, supply chain mis-ordering, etc., a digital twin 130 can be used to model the cart and associated items. Rather than manually completing and tracking preference cards for doctors, nurses, technicians, etc., the digital twin 130 can model, track, simulate, track objects in a surgical field, and predict item usage, user preference, probability of being left behind, etc. Using the “surgical” digital twin 130 results in happier patients at less cost, happier surgeons, nurses and other staff, more savings for healthcare facilities more accurate patient billing, supply chain improvement (e.g., more accurate ordering, etc.), electronic preference card modeling and updating, best practice sharing, etc. Through improved modeling, tracking, predicting/simulating, and reporting via the surgical digital twin 130, re-processing of unused instruments can be reduced, which saves cost in unnecessarily re-purchasing items that were brought into the surgical field but went unused and saved employee time and/or cost in re-processing, for example.

In certain examples, a device, such as an optical head-mounted display (e.g., Google Glass, etc.,) can be used with augmented reality to identify and quantify items (e.g., instruments, products, etc.) in the surgical field, operating room, etc. For example, such a device can be used to validate items selected for inclusion (e.g., on the cart, with respect to the patient, etc.), items used, items tracked, etc., automatically by sight recognition and recording. The device can be used to pull in scanner details from all participants in a surgery, for example, modeled via the digital twin 130 and verified according to equipment list, surgical protocol, personnel preferences, etc.

In certain examples, a “case cart” with prepared materials for a particular case/procedure can be monitored using an optical head-mounted device and/or other technological provided in and/or mounted on the cart, for example. A pick list can be accessible via the cart to identify a patient and applicable supplies for a procedure, for example. The cart and its pick list can be modeled via the digital twin 130, interface with the optical head-mounted device, and/or otherwise be processable to determine item relevance, usage, tracking, disposal/removal, etc.

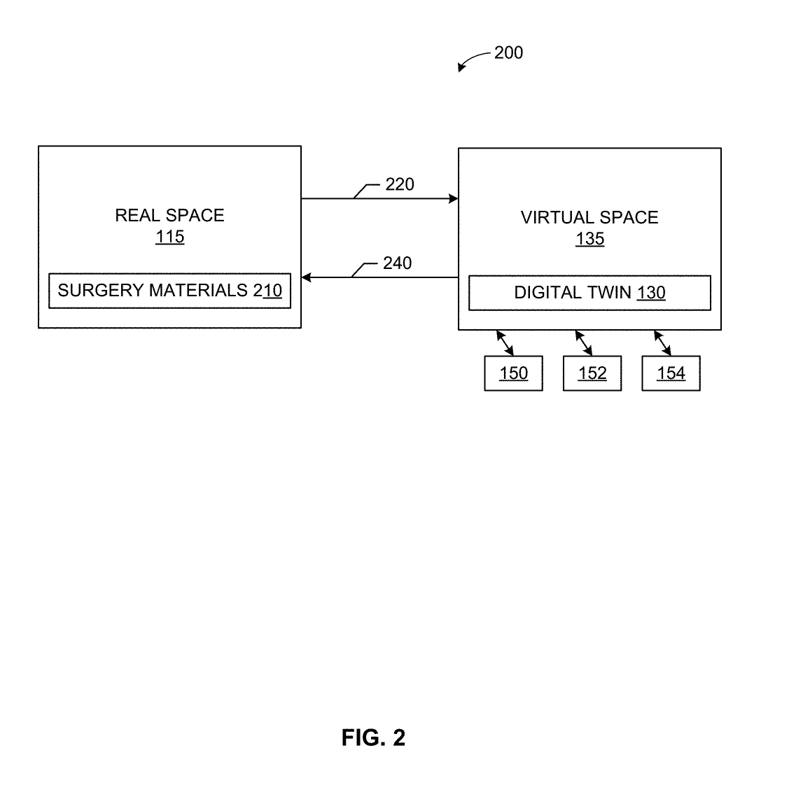

In certain examples, the digital twin 130 can be used to model a preference card and/or other procedure/protocol information for a healthcare user, such as a surgeon, nurse, assistant, technician, administrator, etc. As shown in the example implementation 200 of FIG. 2, surgery materials and/or procedure/protocol information 210 in the real space 115 can be represented by the digital twin 130 in the virtual space 135. Information 220, such as information identifying case/procedure-specific materials, patient data, protocol, etc., can be provided from the surgery materials 210 in the real space 115 to the digital twin 130 in the virtual space 135. The digital twin 130 and/or its virtual space 135 provide information 240 back to the real space 115, for example. The digital twin 130 and/or virtual space 135 can also provide information to one or more virtual sub-spaces 150, 152, 154. As shown in the example of FIG. 2, the virtual space 135 can include and/or be associated with one or more virtual sub-spaces 150, 152, 154, which can be used to model one or more parts of the digital twin 130 and/or digital “sub-twins” modeling subsystems/subparts of the overall digital twin 130. For example, sub-spaces 150, 152, 154 can be used to separately model surgical protocol information, patient information, surgical instruments, pre-operative tasks, post-operative instructions, image information, laboratory information, prescription information, etc. Using the plurality of sources of information, the surgery/operation digital twin 130 can be configured, trained, populated, etc., with patient medical data, exam records, procedure/protocol information, lab test results, prescription information, care plan information, image data, clinical notes, sensor data, location data, healthcare practitioner and/or patient preferences, pre-operative and/or post-operative tasks/information, etc.

When a user (e.g., patient, patient family member (e.g., parent, spouse, sibling, child, etc.), healthcare practitioner (e.g., doctor, nurse, technician, administrator, etc.), other provider, payer, etc.) and/or program, device, system, etc., inputs data in a system such as a picture archiving and communication system (PACS), radiology information system (RIS), electronic medical record system (EMR), laboratory information system (LIS), cardiovascular information system (CVIS), hospital information system (HIS), population health management system (PHM) etc., that information can be reflected in the digital twin 130. Thus, the digital twin 130 can serve as an overall model or avatar of the surgery materials 210 and operating environment 115 in which the surgery materials 210 are to be used and can also model particular aspects of the surgery and/or other procedure, patient care, etc., corresponding to particular data source(s). Data can be added to and/or otherwise used to update the digital twin 130 via manual data entry and/or wired/wireless (e.g., WiFi™, Bluetooth™, Near Field Communication (NFC), radio frequency, etc.) data communication, etc., from a respective system/data source, for example. Data input to the digital twin 130 can be processed by an ingestion engine and/or other processor to normalize the information and provide governance and/or management rules, criteria, etc., to the information. In addition to building the digital twin 130, some or all information can also be aggregated to model user preference, health analytics, management, etc.

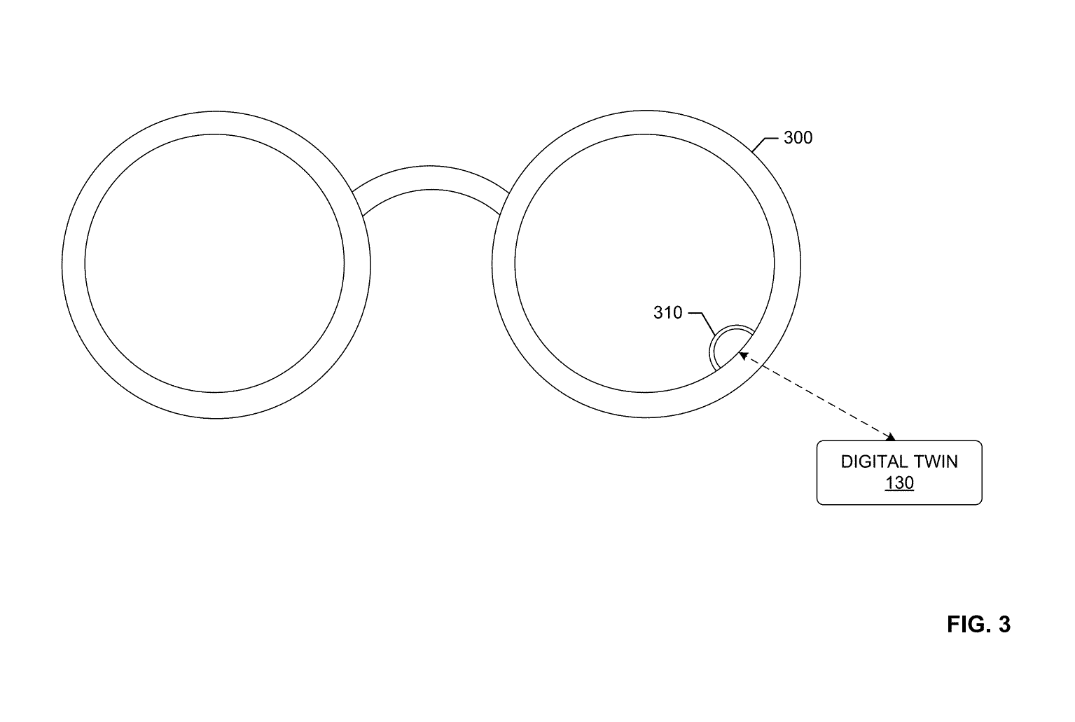

In certain examples, an optical head-mounted display (e.g., Google™ Glass, etc.) can be used to scan and record item such as instruments, instrument trays, disposables, etc., in an operating room, surgical suite, surgical field, etc. As shown in the example of FIG. 3, an optical head-mounted display 300 can include a scanner or other sensor 310 that scans items in its field of view (e.g., scans barcodes, radiofrequency identifiers (RFIDs), visual profile/characteristics, etc.). Item identification, photograph, video feed, etc., can be provided by the scanner 310 to the digital twin 130, for example. The scanner 310 and/or the digital twin 130 can identify and track items within range of the scanner 310, for example. The digital twin 130 can then model the viewed environment and/or objects in the viewed environment based at least in part on input from the scanner 310, for example.

In certain examples, the optical head-mounted display 300 can be constructed using an identifier and counter built into eye shields for instrument(s). Product identifiers can be captured via the scanner 310 (e.g., in an operating room (OR), sterile processing department (SPD), etc.). In certain examples, usage patterns for items can be determined by the digital twin 130 using information captured from the display 300 and its scanner 310. Identified usage patterns can be used by the digital twin 130 and/or connected system(s) to reorder items running low in supply, track items from shipping to receiving to usage location, detect a change in usage pattern, contract status, formulary, etc.

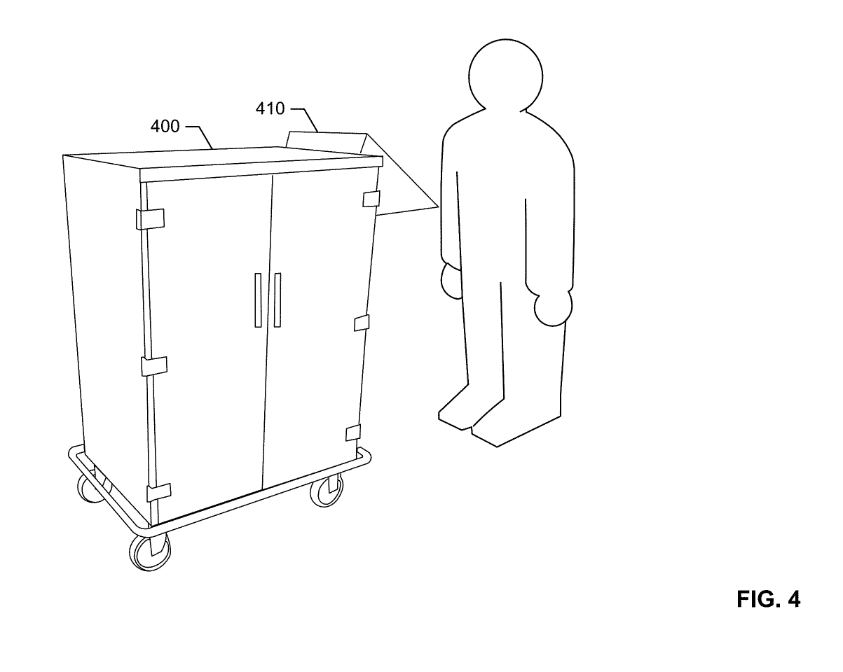

In certain examples, the optical head-mounted display 300 can work alone and/or in conjunction with an instrument cart, such as a surgical cart 400 shown in the example of FIG. 4. The example surgical cart 400 can include a computing device 410, such as a tablet computer and/or other computing interface to receive input from a user and providing output regarding content of the cart 400, associated procedure/protocol, other user(s) (e.g., patient, healthcare practitioner(s), etc.), instrument usage for a procedure, etc. The computing device 410 can be used to house the surgical digital twin 130, update and/or otherwise communicate with the digital twin 130, store preference card(s), store procedure/protocol information, track protocol compliance, generate analytics, etc.

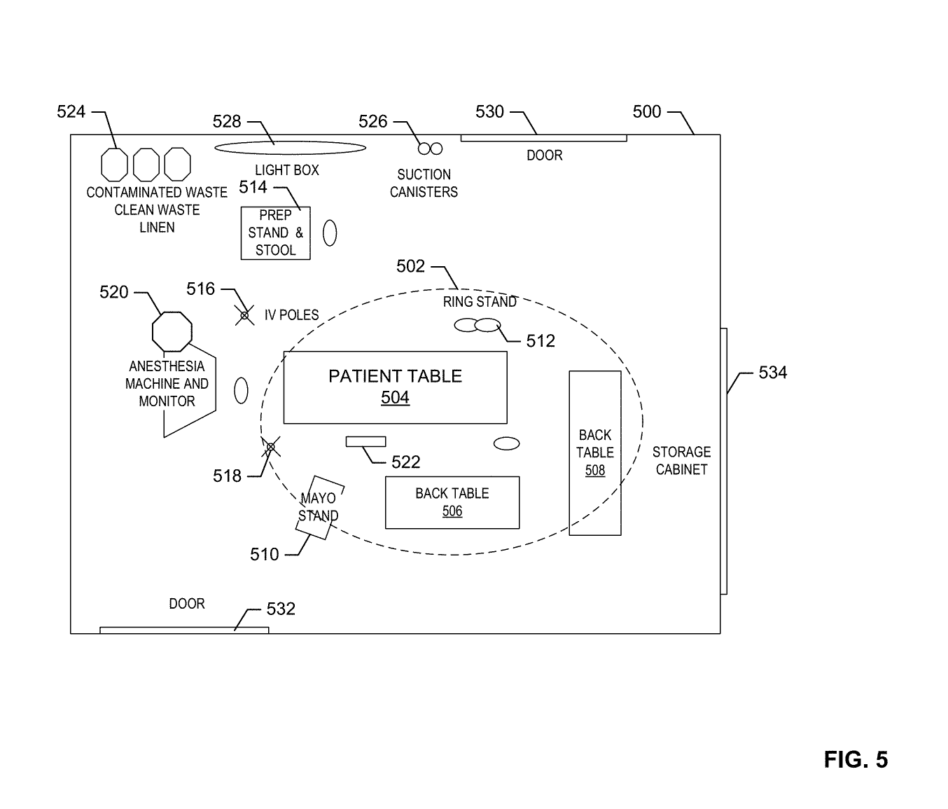

FIG. 5 illustrates an example monitored environment 500 for the digital twin 130. The example environment 500 (e.g., operating room, surgical suite, etc.) includes a sterile field 502 and a patient table 504. The example environment 500 also includes one or more additional tables 506, 508, stands 510, 512, 514, light box 516, intravenous (IV) fluid poles 516, 518, etc., inside and/or outside the sterile field 502. The example environment 500 can also include one or more machines such as an anesthesia machine and monitor 520. The example environment 500 can include one or more steps 522, one or more containers 524 for contaminated waste, clean waste, linen, etc. The example environment 500 can include one or more suction canisters 526, light box 528, doors 530, 532, storage 534, etc. The example optical head-mounted display 300 and/or the example cart 400 can be in the environment 500 and can scan and/or otherwise gather input from objects (e.g., people, resources, other items, etc.) in the environment 500 (e.g., in the sterile field 502) and can generate report(s), import information into the digital twin 130, etc.

For example, within the surgical field 502, a scrub nurse may stand on the step 522 during a procedure. The back table 506, 508 has products opened for the procedure. Open products can include hundreds of items and instruments, necessitating an automatic way of scanning, updating, and modeling the environment 500. Under certain guidelines (e.g., professional guidelines such as Association of periOperative Registered Nurses (AORN) guidelines, etc.), recommended maximum weight for instrument trays is 18 pounds. However, a procedure can involve multiple instrument trays. When an instrument tray is opened, all instruments on the tray have to be reprocessed, whether or not they were used. For example, all instruments are required to be decontaminated, put back in stringers, re-sterilized, etc.

Using the optical head-mounted display 300 and/or the cart 400, instrument tray(s) can be automatically scanned from the table(s) 504-508, stand(s) 510-514, etc. Thus, instruments in the example environment 500 (e.g., within the surgical field 502, etc.) can be automatically measured to improve tracking and patient safety as well as to save on reprocessing costs and resupply costs, for example. Information regarding the instrument tray(s), associated procedure(s), patient, healthcare personnel, etc., can be provided to the digital twin 130 via the head-mounted display 300 and/or cart 400 to enable the digital twin 130 to model conditions in the example environment 500 including the surgical field 502, patient table 504, back table(s) 506, 508 stand(s) 510-514, pole(s) 516-518, monitor(s) 520, step(s) 522, waste/linen container(s) 524, suction canister(s) 526, light box 528, door(s) 530-532, storage cabinet 534, etc.

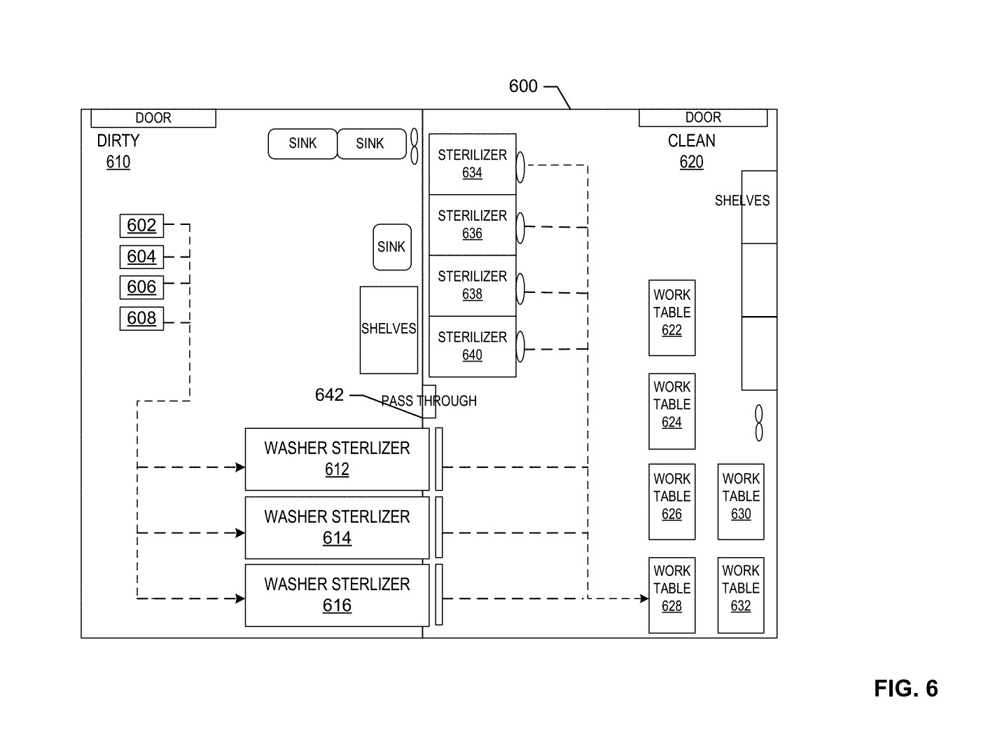

FIG. 6 illustrates an example instrument processing facility 600 for processing/re-processing instruments (e.g., surgical instruments, etc.). For example, the facility 600 can process instruments through a plurality of steps or elements beginning with dirty to decontaminate, clean, inspect, reassemble (e.g., process, position, and re-wrap, etc.), and sterilized to be used for another procedure. As shown in the example of FIG. 6, one or more case carts 602-608 (e.g., same or similar to the instrument cart 400 of the example of FIG. 4, etc.) are brought into the dirty portion 610 of the processing facility 600. The carts 602-608 and/or items on the carts 602-608 (e.g., surgical instruments, leftover implants, etc.) can be provided to one or more washer sterilizers 612-616 in a clean section 620 of the processing facility 600. After passing through the sterilizers 612-616, the item(s) can be placed on work table(s) 622-632 in the clean portion 620 of the processing facility 600. Additional sterilizers 634-640 in the clean portion 620 can sterilize additional items in preparation for packaging, arrangement, etc., for use in a procedure, etc. A pass-through 642 allows for personnel, item(s), cart(s), etc., to pass from the dirty side 610 to the clean side 620 of the processing facility 600. Items such as instrument(s), cart(s), etc., can be scanned in the dirty section 610, clean section 620, prior to sanitization, during sanitization, after sanitization, etc., via the optical head-mounted display 300 and/or the cart tablet 410, for example, and provided to the surgical digital twin 130. For example, items can be tracked and deficiencies such as chips in stainless/sterile coatings, foreign substances, and/or insufficient cleaning be identified using the device(s) 300, 410, etc.

In certain examples, the device 300 and/or 410 can provide a display window including information regarding instruments, protocol actions, implants, items, etc. For example, the display window can include information regarding costs associated with the trash, including information regarding supply utilization and costs associated therewith. The display window can include information regarding the surgery being performed on the patient, including descriptive information about the surgery, and financial performance information, for example.

In certain examples, alternatively or in addition to scanning provided by the scanner 310 and/or the computing device 410, voice recognition/control can be provided in the environment 500 and/or 600. In certain examples, an audio capture and/or other voice command/control device (e.g., Amazon Echo™, Google Home™, etc.) can capture a conversation and assign a verbal timestamp. The device can ask questions and provide information, for example. For example, the device can detect a spoken command to “This is room five, and I need more suture” and can automatically send a message to provide a suture to room five. For example, in the perioperative space, the voice-activated communication device can be triggered to record audio (e.g., conversation, patient noises, background noise, etc.) during a pre-operative (“pre-op”) period (e.g., sign-in, data collection, etc.). On the day of surgery (DOS), a pre-op sign-in process can include voice recording of events/nursing, documentation and throughput indicators, etc. In a post-operative (post-op) period, a follow-up survey can be voice recorded, for example. In certain examples, the voice-activated communication device can serve as a virtual assistant to help the healthcare user, etc.

In certain examples, the voice-activated communication device can be paired with a projector and/or other display device to display information, such as a voice-activated white board, voice-activated computing device 410, voice activated device 300, etc.

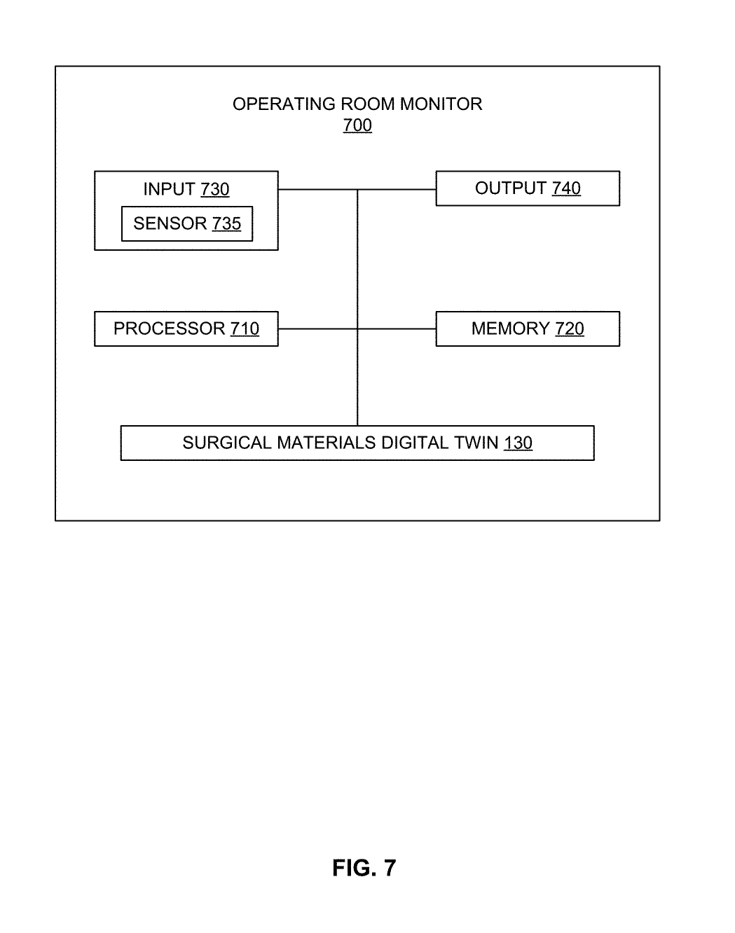

FIG. 7 illustrates an example operating room monitor 700 including a processor 710, a memory 720, an input 730, an output 740, and a surgical materials digital twin 130. The example input 730 can include a sensor 735, for example. The sensor 735 can monitor items, personnel, activity, etc., in an environment 500, 600 such as an operating room 500.

For example, the sensor 735 can detect items on the table(s) 504-508, status of the patient on the patient table 504, position of stand(s) 510-514, pole(s) 516-518, monitor 520, step 522, waste/linen 524, canisters 526, door(s) 528-530, storage 532, etc. As another example, the sensor 735 can detect cart(s) 602-608 and/or item(s) on/in the cart(s) 602-608. The sensor 735 can detect item(s) on/in the sterilizer(s) 612-640, on table(s) 622-632, in the pass-through 642, etc. Object(s) detected by the sensor 735 can be provided as input 730 to be stored in memory 720 and/or processed by the processor 710, for example. The processor 710 (and memory 720) can update the surgical materials digital twin 130 based on the object(s) detected by the sensor 735 and identified by the processor 710, for example.

In certain examples, the digital twin 130 can be leveraged by the processor 710, input 730, and output 740 to provide a simulation in preparation for and/or follow-up to a surgical procedure. For example, the surgical materials digital twin 130 can model items including the cart 400, surgical instruments, implant and/or disposable material, etc., to be used by a surgeon, nurse, technician, etc., to prepare for the procedure. The modeled objects can be combined with procedure/protocol information (e.g., actions/tasks in the protocol correlated with associated item(s), etc.) to guide a healthcare practitioner through a procedure and/or other protocol flow (e.g., mySurgicalAssist), for example. Potential outcome(s), possible emergency(-ies), impact of action/lack of action, etc., can be simulated using the surgical digital twin 130, for example.

In certain examples, the operating room monitor 700 can help facilitate billing and payment through modeling and prediction of charges associated with events (e.g., protocol steps, surgical materials, etc.), etc. For example, the digital twin 130 can evaluate which items and actions will be used in a surgical procedure as well as a cost/charge associated with each item/action. The digital twin 130 can also model insurance and/or other coverage of resources and can combine the resource usage (e.g., personnel time/action, material, etc.) with cost and credit/coverage/reimbursement to determine how and who to bill and collect from in what amount(s) for which charge(s), for example. Thus, not only can the monitor 700 and its surgical assist digital twin 130 help a surgeon and/or other healthcare personnel plan for a surgical procedure, the monitor 700 and its digital twin 130 can help administrative and/or other financial personnel bill and collect for that surgical procedure, for example.

In certain examples, the monitor 700 and its digital twin 130 and processor 710 can facilitate bundled payment. For example, rather than independent events, several events may be included in an episode of care (e.g., a preoperative clinic for lab work, preoperative education, surgical operation, post-operative care, rehabilitation, etc.). The digital twin 130 can model and organize (e.g., bundle) the associated individual payments into one bundled payment for a hospital and/or other healthcare institution, for example.

The digital twin 130 (e.g., with input 730 and output 740, processor 710, memory 720, etc.) can also provide a compliance mechanism to motivate people to continue and comply with preop care, postop follow-up, payment, rehab, etc. For example, the digital twin 130 can be leveraged to help prompt, track, incentivize, and analyze patient rehab in between physical therapy appointments to help ensure compliance, etc. For example, the input 730 can include a home monitor such as a microphone, camera, robot, etc., to monitor patient activity and compliance for the digital twin 130, and the output 740 can include a speaker, display, robot, etc., to interact with the patient and respond to their activity/behavior. Thus, the digital twin 130 can be used to engage the patient 110 before a procedure, during the procedure, and after the procedure to promote patient care and wellness, for example. The monitor 700 and digital twin 130 can be used to encourage patient and provider engagement, interaction, ownership, etc. The digital twin 130 can also be used to help facilitate workforce management to model/predict a care team and/or other personnel to be involve in preop, operation, postop, follow-up, etc., for one or more patients, one or more procedures, etc. The digital twin 130 can be used to monitor, model, and drive a patient's journey from patient monitoring, virtual health visit, in-person visit, treatment, postop monitoring, social/community engagement, etc.

In certain examples, the monitor 700 can be implemented in a robot, a smart watch, the optical display 300, the cart tablet 410, etc., which can be connected in communication with an electronic medical record (EMR) system, picture archiving and communication system (PACS), radiology information system (RIS), archive, imaging system, etc. Certain examples can facilitate non-traditional partnerships, different partnership models, different resource usage (e.g., precluding use of prior resources already used in a linear care path/curve, etc.), etc.

Certain examples leverage the digital twin 130 to help prevent postoperative complications such as those that may result in patient readmission to the hospital and/or surgical center. The digital twin 130 can model likely outcome(s) given input information regarding patient, healthcare practitioner(s), instrument(s), other item(s), procedure(s), etc., and help the patient and/or an associated care team to prepare and/or treat the patient appropriately to avoid/head off undesirable outcome(s), for example.

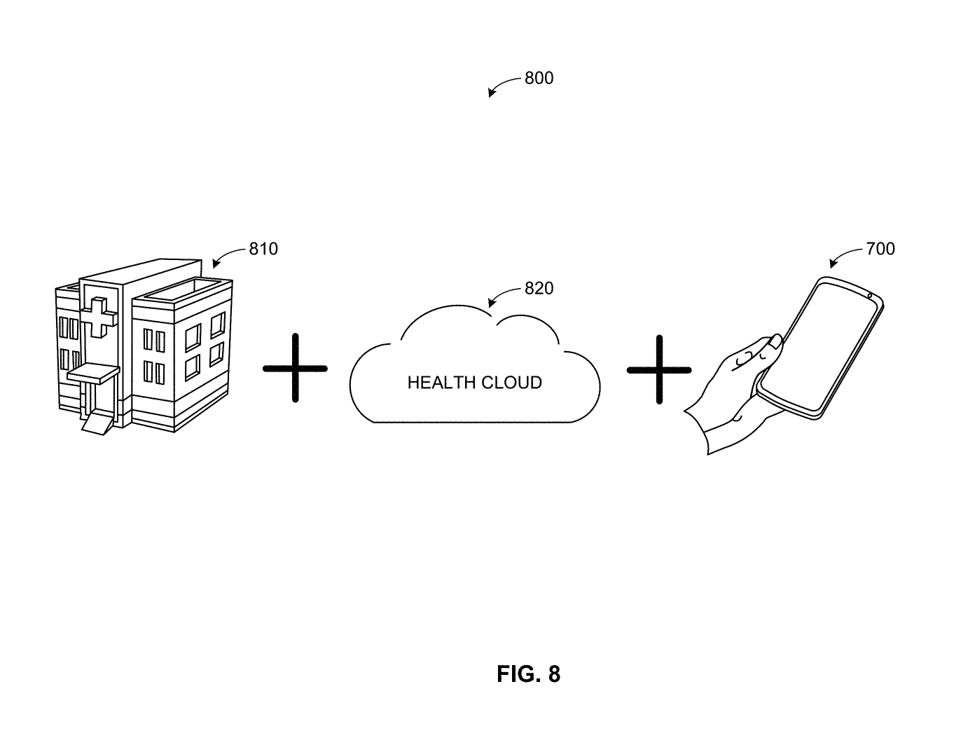

Thus, as illustrated in the example ecosystem 800 of FIG. 8, the example monitor 700 can work with one or more healthcare facilities 810 via a health cloud 820 to facilitate trending and tracking of surgical procedures and other protocol compliance via the digital twin 130. The digital twin 130 can be stored at the monitor 700, healthcare facility 810, and/or health cloud 820, for example. The digital twin 130 can model healthcare practitioner preference, patient behavior/response with respect to a procedure, equipment usage before/during/after a procedure, etc., to predict equipment needs, delays, potential issues with patient/provider/equipment, possible complication(s), etc. Alphanumeric data, voice response, video input, image data, etc., can provide a multi-media model of a procedure to the healthcare practitioner, patient, administrator, insurance company, etc., via the patient digital twin 130, for example.

In certain examples, matching pre-op data, procedure data, post-op data, procedure guidelines, patient history, practitioner preferences, and the digital twin 130 can identify potential problems for a procedure, item tracking, and/or post-procedure recovery and develop or enhance smart protocols for recovery crafted for the particular procedure, practitioner, facility, and/or patient, for example. The digital twin 130 continues to learn and improve as it receives and models feedback throughout the pre-procedure, during procedure, and post-procedure process including information regarding items used, items unused, items left, items missing, items broken, etc.

In certain examples, improved modeling of a procedure via the digital twin 130 can reduce or avoid post-op complications and/or follow-up visits. Instead, preferences, reminders, alerts, and/or other instructions, as well as likely outcomes, can be provided via the digital twin 130. Through digital twin 130 modeling, simulation, prediction, etc., information can be communicated to practitioner, patient, supplier, insurance company, administrator, etc., to improve adherence to pre- and post-op instructions and outcomes, for example. Feedback and modeling via the digital twin 130 can also impact the care provider. For example, a surgeon's preference cards can be updated/customized for the particular patient and/or procedure based on the digital twin 130. Implants, such as knee, pacemaker, stent, etc., can be modeled for the benefit of the patient and the provider via the digital twin 130, for example. Instruments and/or other equipment used in procedures can be modeled, tracked, etc., with respect to the patient and the patient's procedure via the digital twin 130, for example. Alternatively or in addition, parameters, settings, and/or other configuration information can be pre-determined for the provider, patient, and a particular procedure based on modeling via the digital twin 130, for example.

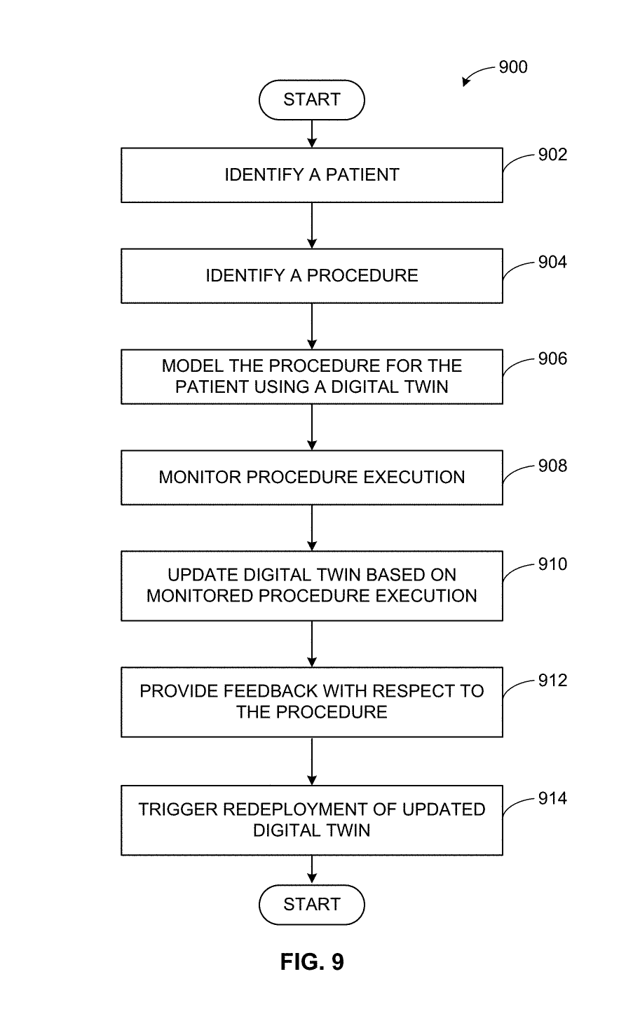

FIG. 9 illustrates an example process 900 for procedure modeling using the digital twin 130. At block 902, a patient is identified. For example, a patient on which a surgical procedure is to be performed is identified by the monitor 700 and modeled in the digital twin 130 (e.g., based on input 730 information such as EMR information, lab information, image data, scheduling information, etc.). At block 904, a procedure and/or other protocol is identified. For example, a knee replacement and/or other procedure can be identified (e.g., based on surgical order information, EMR data, scheduling information, hospital administrative data, etc.).

At block 906, the procedure is modeled for the patient using the digital twin 130. For example, based on the identified procedure, the digital twin 130 can model the procedure to facilitate practice for healthcare practitioners to be involved in the procedure, predict staffing and care team make-up associated with the procedure, improve team efficiency, improve patient preparedness, etc. At block 908, procedure execution is monitored. For example, the monitor 700 including the sensor 735, optics 300, tablet 410, etc., can be used to monitor procedure execution by detecting object position, time, state, condition, and/or other aspect to be modeled by the digital twin 130.

At block 910, the digital twin 130 is updated based on the monitored procedure execution. For example, the object position, time, state, condition, and/or other aspect captured by the sensor 735, optics 300, tablet 410, etc., is provided via the input 730 to be modeled by the digital twin 130. A new model can be created and/or an existing model can be updated using the information. For example, the digital twin 130 can include a plurality of models or twins focusing on particular aspects of the environment 500, 600 such as surgical instruments, disposables/implants, patient, surgeon, equipment, etc. Alternatively or in addition, the digital twin 130 can model the overall environment 500, 600.

At block 912, feedback is provided with respect to the procedure. For example, the digital twin 130 can work with the processor 710 and memory 720 to generate an output 740 for the surgeon, patient, hospital information system, etc., to impact conducting of the procedure, post-operative follow-up, rehabilitation plan, subsequent pre-operative care, patient care plan, etc. The output 740 can warn the surgeon, nurse, etc., that an item is in the wrong location, is running low/insufficient for the procedure, etc., for example. The output 740 can provide billing for inventory and/or service, for example, and/or update a central inventory based on item usage during a procedure, for example.

At block 914, periodic redeployment of the updated digital twin 130 is triggered. For example, feedback provided to and/or generated by the digital twin 130 can be used to update a model forming the digital twin 130. When a certain threshold of new data is reached, for example, the digital twin 130 can be retrained, retested, and redeployed to better mimic real-life surgical procedure information including items, instruments, personnel, protocol, etc. In certain examples, updated protocol/procedure information, new best practice, new instrument and/or personnel, etc., can be provided to the digital twin 130, resulting in an update and redeployment of the updated digital twin 130. Thus, the digital twin 130 and the monitor 700 can be used to dynamically model, monitor, train, and evolve to support surgery and/or other medical protocol, for example.

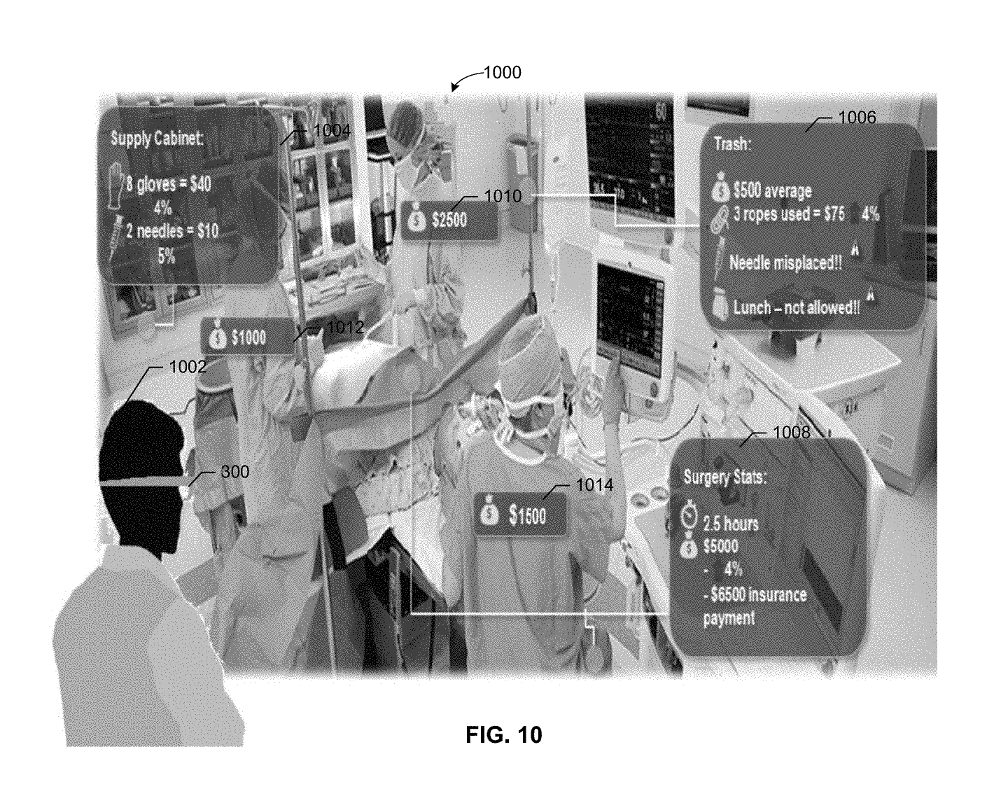

In certain examples, such as FIG. 10, information from the digital twin 130 can be provided via augmented reality (AR) such as via the glasses 300 to a user, such as a surgeon, etc., in the operating room. FIG. 10 presents an example AR visualization 1000 including auxiliary information regarding various aspects of an operating room environment in accordance with one or more embodiments described herein. One or more aspects of the example AR visualization 1000 demonstrate the features and functionalities of systems 100-800 (and additional systems described herein) with respect to equipment/supplies assessment and employee assessment, for example.

The example AR visualization 1000 depicts an operating room environment (e.g., 500) of a healthcare facility that is being viewed by a user 1002. The environment includes three physicians operating on a patient. In the embodiments shown, the user 1002 is wearing an AR device 300 and physically standing in the healthcare facility with a direct view of the area of the operating room environment viewed through transparent display of the AR device 300. However, in other implementations, the user 1002 can be provided at a remote location and view image/video data of the area and/or model data of the area on a remote device. In certain examples, the AR device 300 can include or be communicatively coupled to an AR assistance module to facilitate providing the user with auxiliary information regarding usage and/or performance of a healthcare system equipment in association with viewing the equipment.

The example AR visualization 1000 further includes overlay data including information associated with various supplies, equipment and people (e.g., the physicians and the patient) included in the operating room 500 such as determined by the sensor 310, for example. Example information represented in the overlay data includes utilization and performance information associated with the various supplies, equipment and people, that have been determined to be relevant to the context of the user 1002. For example, display window 1004 includes supply utilization information regarding gloves and needles in the supply cabinet. Display window 1004 also includes financial performance information regarding costs attributed to the gloves and needles. Display window 1006 includes information regarding costs associated with the trash, including information regarding supply utilization and costs associated therewith. Display window 1008 includes information regarding the surgery being performed on the patient, including descriptive information about the surgery, and financial performance information. Further, the overlay data includes display windows 1010, 1012, and 1014 respectively providing cost information regarding cost attributed to the utilization of the respective physicians for the current surgery. As with the other visualizations described herein, it should be appreciated that the appearance and location of the overlay data (e.g., display windows 1004-1014) in the example visualization 1000 are merely examples and intended to convey the concept of what is actually viewed by the user through the AR device 300. However, the appearance and location of the overlay data in visualization 1000 is not technically accurate, as the actual location of the overlay data would be on the glass/display of the AR device 300. Additionally, in certain examples, the user 1002 can control the AR device 300 through motions, buttons, touches, etc., to show, edit, and/or otherwise change the AR display, and the sensor 310 can detect and react to user control commands/actions/gestures.

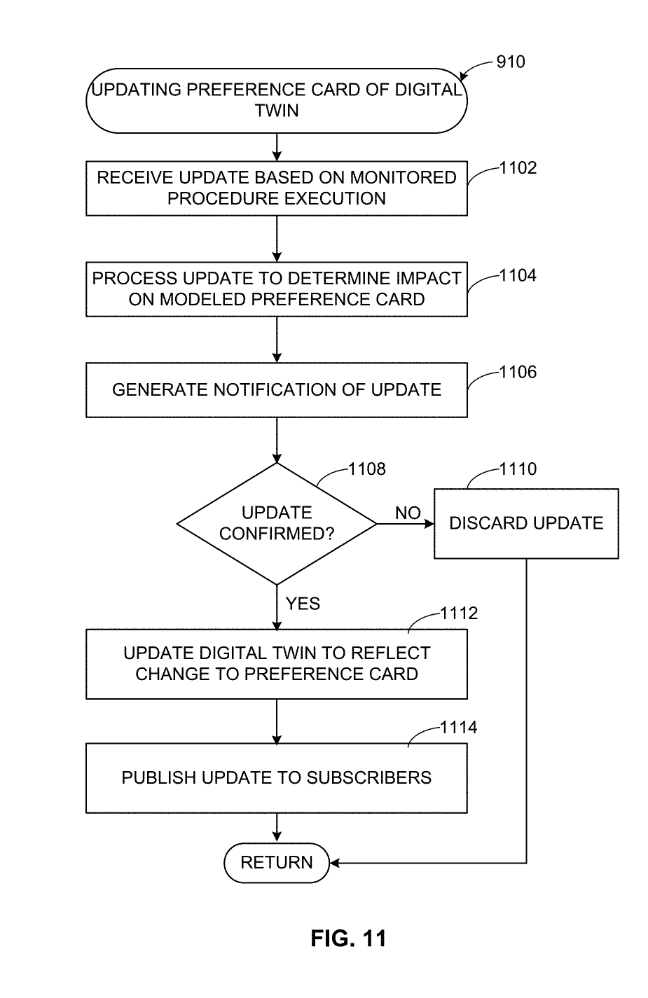

FIG. 11 provides further detail regarding updating of the digital twin 130 including a preference card based on monitored procedure execution (block 910). At block 1102, the digital twin 130 receives an update based on the monitored execution of the procedure and/or other protocol. The update includes monitored execution information including tools and/or other items used in the procedure, implants and/or disposables used in the procedure, protocol actions associated with the procedure, personnel involved in the procedure, etc.

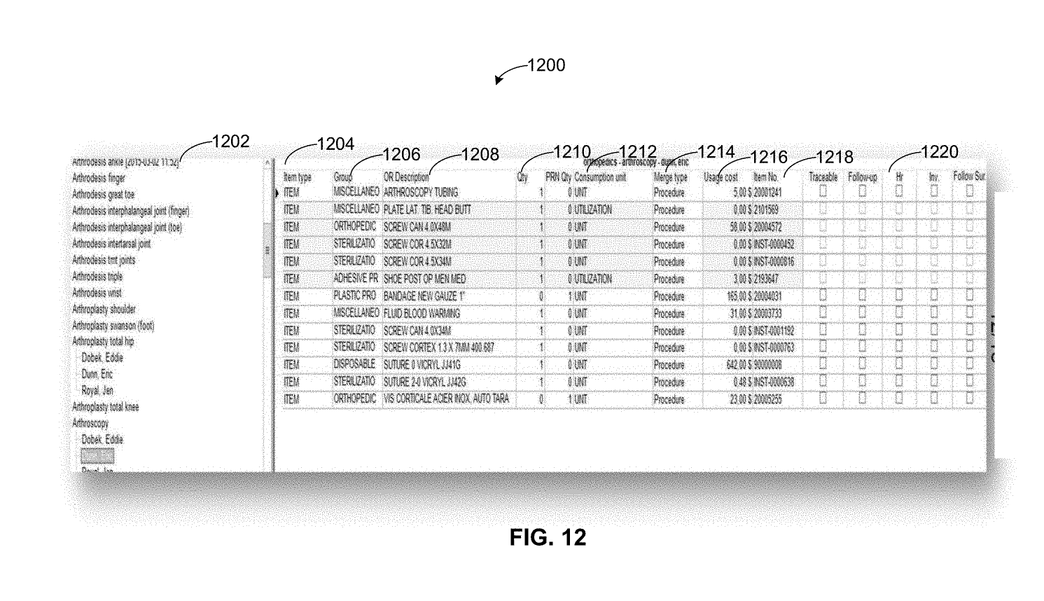

At block 1104, the update is processed to determine its impact on the modeled preference card of the digital twin 130. For example, a preference card can provide a logical set of instructions for item and personnel positioning for a surgical procedure, equipment and/or other supplies to be used in the surgical procedure, staffing, schedule, etc., for a particular surgeon, other healthcare practitioner, surgical team, etc. The digital twin 130 can model one or more preference cards including to update the preference card(s), simulate using the preference card(s), predict using the preference card(s), train using the preference card(s), analyze using the preference card(s), etc. FIG. 12 illustrates an example preference card 1200 for an arthroscopic orthopedic procedure.

As shown in the example of FIG. 12, the preference card 1200 includes a plurality of fields to identify information, provide parameters, and/or set other preferences for a surgical procedure by user. For example, the preference card 1200 includes a list 1202 organized by procedure and/or user. For each item in the list 1202, one or more items preferred by the user and/or best practice for the procedure are provided by item type 1204, associated group 1206, and description 1208. A quantity 1210, unit of consumption 1212, merge type 1214, usage cost 1216, and item number 1218 can also be provided to allow the digital twin 130 to model and plan, order, configure, etc., the items for a procedure. The modeled procedure card 1200 can also include one or more fields to indicate traceability, follow-up, etc.

At block 1106, a user, application, device, etc., is notified of the update. For example, a message regarding the update and an indication of the impact of the update on the modeled preference card of the digital twin 130 are generated and provided to the user (e.g., a surgeon, nurse, other healthcare practitioner, administrator, supplier, etc.), application (e.g., scheduling application, ordering/inventory management application, radiology information system, practice management application, electronic medical record application, etc.), device (e.g., cart tablet 410, optical device 300, etc.), etc.

At block 1108, input is processed to determine whether the update is confirmed. For example, via the glasses 300, tablet 410, and/or other device (such as via the input 730 of the monitor 700) the user and/or other application, device, etc., can confirm or deny the update to the preference card of the digital twin 130. For example, a surgeon associated with the modeled preference card 1200 can review and approve or deny the update/change to the modeled preference card 1200. At block 1110, if the update is not confirmed, then the change to the preference card model is reversed and/or otherwise discarded. However, at block 1112, if the update is confirmed, then the digital twin 130 is updated to reflect the change to the preference card 1200 modeled by the digital twin 130.

At block 1114, the update is published to subscriber(s). For example, digital twin subscribers, preference card subscribers, etc., can receive a notice regarding the preference card update, a copy of the updated preference card model, etc.

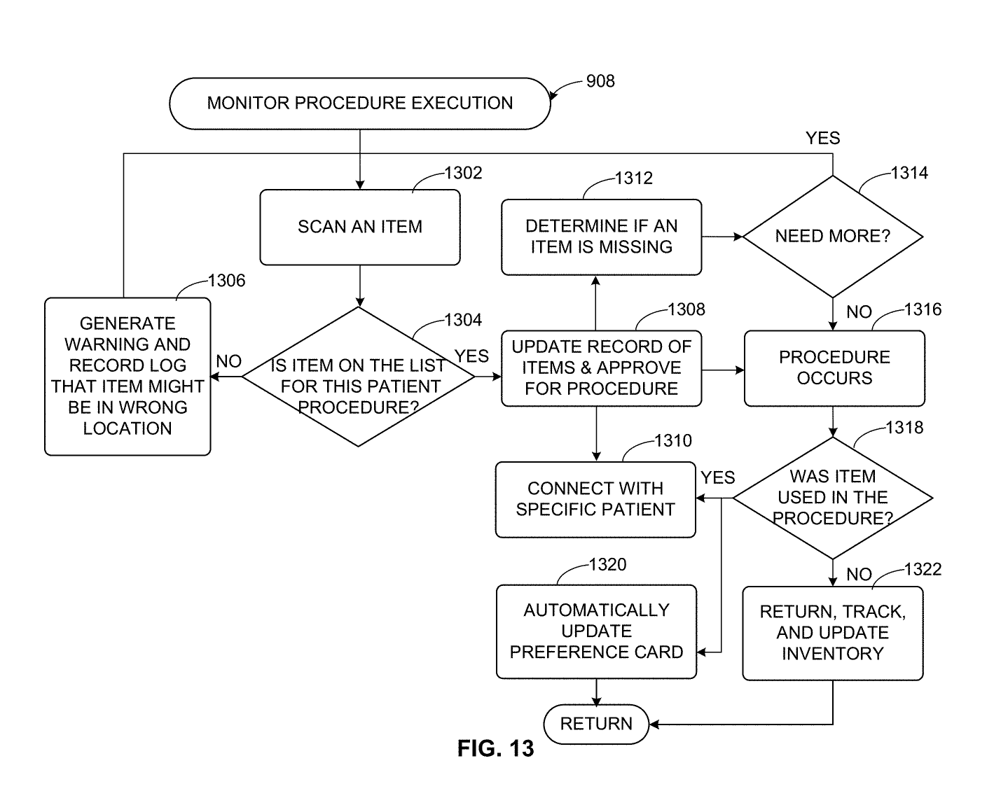

FIG. 13 illustrates an example implementation of monitoring procedure execution (block 908). At block 1302, an item is scanned, such as by the scanner 310 of the optical glasses 300, eye shield, etc. For example, object recognition, bar code scan, etc., can be used to identify the item.

At block 1304, the scanned item is evaluated to determine whether it is included in a list or set of items for the procedure for the patient (e.g., on the preference card 1200 and/or otherwise included in the protocol and/or best practices for the procedure, etc.). At block 1306, if the item is not on the list for the particular patient's procedure, then a warning is generated and logged to indicate that the item might be in the wrong location. For example, if the wrong implant is scanned in the operating room, the implant is flagged as not included on the procedure list for the patient, and the surgeon and/or other healthcare practitioner is alerted to warn them of the presence of the wrong implant for the procedure.

At block 1308, if the item is on the list for the patient's procedure, then a record of items for the procedure is updated, and the item is approved for the procedure. For example, if the implant is approved for the particular patient's surgery, the presence of the implant is recorded, and the implant is approved for insertion into the patient in the surgery. At block 1310, the item is connected with the particular patient undergoing the procedure. Thus, for example, the item can be added to the patient's electronic medical record, invoice/bill, etc.

At block 1312, the record of items for the procedure is evaluated by the digital twin 130 (e.g., by the processor 710 using the model of the digital twin 130) to identify missing item(s). For example, the record of items is compared to a modeled list of required items, preferred items, suggested items, etc., to identify item(s) that have not yet been scanned and recorded for the procedure. At block 1314, missing item(s) are evaluated. If more item(s) are to be included, then control reverts to block 1302 to scan another item. If items are accounted for, then control moves to block 1316, during which the procedure occurs for the patient. The procedure is monitored to update the digital twin 130 and/or otherwise provide feedback, for example.

At block 1318, item(s) are analyzed to determine whether the item(s) were used in the procedure. If an item was used in the procedure, then, at block 1310, the item can be connected with the patient record. Use of the item also triggers, at block 1320, an automatic update of the preference card (e.g., at the digital twin 130, etc.).

If the item was not used in the procedure, then, at block 1322, the item is returned to the cart 400, tracked, and updated with respect to the central inventory to account for the item remaining after the procedure. Thus, if the item was used in the patient's surgery, the preference card and other record(s) can be updated to reflect that use. If the item was not used, then the patient does not need to be billed for the item and then item may not be listed on the preference card for that surgeon for the given procedure, for example.

Thus, for example, Doctor Jones is very consistent about his preferences for his procedures. However, at some point he changes from using product X to using product Y such that a preference card associated with Doctor Jones is now incorrect. Using the digital twin 130 and the method 900, the preference card 1200 for Doctor Jones can be updated to reflect the usage of product Y for one or more procedures. The system sends an email, message, and/or other notice to Doctor Jones for Doctor Jones to confirm the potential preference change. Doctor Jones can confirm or deny the change, and the preference card 1200 modeled in the digital twin 130 can be adjusted accordingly. Doctor Jones can also provide an explanation or other understanding of why he changed from product X to product Y. The digital twin 130 can then share the understanding of why the decision to change was made with other subscribing practitioners (e.g., surgeons, nurses, etc.), for example.

Machine Learning Examples

Machine learning techniques, whether deep learning networks or other experiential/observational learning system, can be used to model information in the digital twin 130 and/or leverage the digital twin 130 to analyze and/or predict an outcome of a procedure, such as a surgical operation and/or other protocol execution, for example. Deep learning is a subset of machine learning that uses a set of algorithms to model high-level abstractions in data using a deep graph with multiple processing layers including linear and non-linear transformations. While many machine learning systems are seeded with initial features and/or network weights to be modified through learning and updating of the machine learning network, a deep learning network trains itself to identify “good” features for analysis. Using a multilayered architecture, machines employing deep learning techniques can process raw data better than machines using conventional machine learning techniques. Examining data for groups of highly correlated values or distinctive themes is facilitated using different layers of evaluation or abstraction.

Deep learning is a class of machine learning techniques employing representation learning methods that allows a machine to be given raw data and determine the representations needed for data classification. Deep learning ascertains structure in data sets using backpropagation algorithms which are used to alter internal parameters (e.g., node weights) of the deep learning machine. Deep learning machines can utilize a variety of multilayer architectures and algorithms. While machine learning, for example, involves an identification of features to be used in training the network, deep learning processes raw data to identify features of interest without the external identification.

Deep learning in a neural network environment includes numerous interconnected nodes referred to as neurons. Input neurons, activated from an outside source, activate other neurons based on connections to those other neurons which are governed by the machine parameters. A neural network behaves in a certain manner based on its own parameters. Learning refines the machine parameters, and, by extension, the connections between neurons in the network, such that the neural network behaves in a desired manner.

Deep learning that utilizes a convolutional neural network (CNN) segments data using convolutional filters to locate and identify learned, observable features in the data. Each filter or layer of the CNN architecture transforms the input data to increase the selectivity and invariance of the data. This abstraction of the data allows the machine to focus on the features in the data it is attempting to classify and ignore irrelevant background information.

Alternatively or in addition to the CNN, a deep residual network can be used. In a deep residual network, a desired underlying mapping is explicitly defined in relation to stacked, non-linear internal layers of the network. Using feedforward neural networks, deep residual networks can include shortcut connections that skip over one or more internal layers to connect nodes. A deep residual network can be trained end-to-end by stochastic gradient descent (SGD) with backpropagation, for example.

Deep learning operates on the understanding that many datasets include high level features which include low level features. While examining an image of an item in the surgical field 502, for example, rather than looking for an object, it is more efficient to look for edges which form motifs which form parts, which form the object being sought. These hierarchies of features can be found in many different forms of data such as speech and text, etc.

Learned observable features include objects and quantifiable regularities learned by the machine during supervised learning. A machine provided with a large set of well classified data is better equipped to distinguish and extract the features pertinent to successful classification of new data.

A deep learning machine that utilizes transfer learning may properly connect data features to certain classifications affirmed by a human expert. Conversely, the same machine can, when informed of an incorrect classification by a human expert, update the parameters for classification. Settings and/or other configuration information, for example, can be guided by learned use of settings and/or other configuration information, and, as a system is used more (e.g., repeatedly and/or by multiple users), a number of variations and/or other possibilities for settings and/or other configuration information can be reduced for a given situation.

An example deep learning neural network can be trained on a set of expert classified data, for example. This set of data builds the first parameters for the neural network, and this would be the stage of supervised learning. During the stage of supervised learning, the neural network can be tested whether the desired behavior has been achieved.

Once a desired neural network behavior has been achieved (e.g., a machine has been trained to operate according to a specified threshold, etc.), the machine can be deployed for use (e.g., testing the machine with “real” data, etc.). During operation, neural network classifications can be confirmed or denied (e.g., by an expert user, expert system, reference database, etc.) to continue to improve neural network behavior. The example neural network is then in a state of transfer learning, as parameters for classification that determine neural network behavior are updated based on ongoing interactions. In certain examples, the neural network can provide direct feedback to another process. In certain examples, the neural network outputs data that is buffered (e.g., via the cloud, etc.) and validated before it is provided to another process.

Deep learning machines using convolutional neural networks (CNNs) can be used for data analysis. Stages of CNN analysis can be used for facial recognition in natural images, computer-aided diagnosis (CAD), object identification and tracking, etc.

Deep learning machines can provide computer aided detection support to improve item identification, relevance evaluation, and tracking, for example. Supervised deep learning can help reduce susceptibility to false classification, for example. Deep learning machines can utilize transfer learning when interacting with physicians to counteract the small dataset available in the supervised training. These deep learning machines can improve their protocol adherence over time through training and transfer learning.

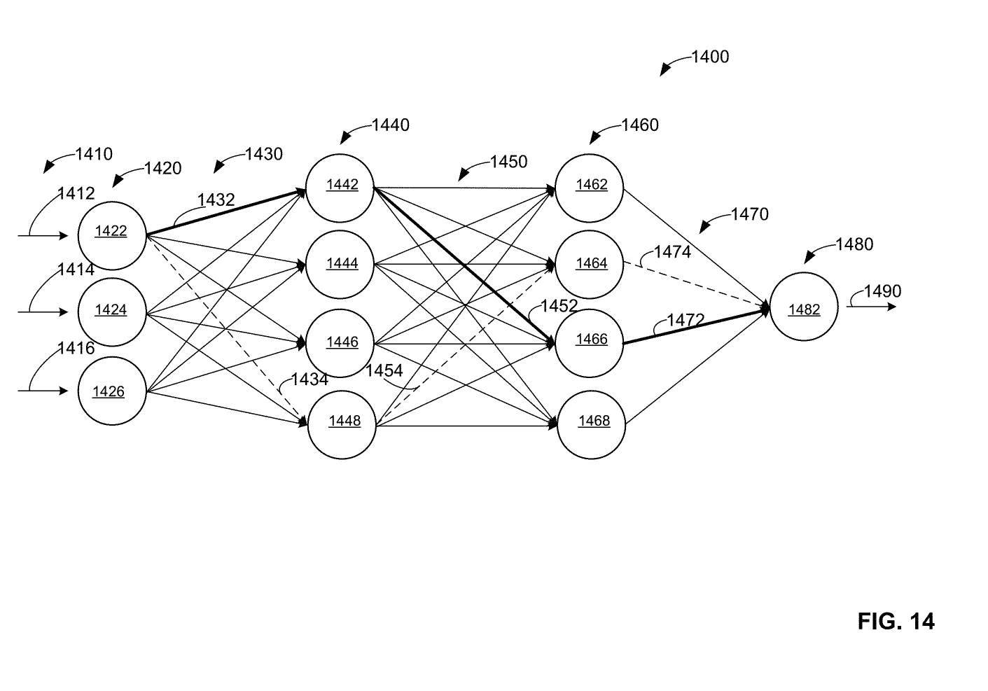

FIG. 14 is a representation of an example deep learning neural network 1400 that can be used to implement the surgery digital twin 130. The example neural network 1400 includes layers 1420, 1440, 1460, and 1480. The layers 1420 and 1440 are connected with neural connections 1430. The layers 1440 and 1460 are connected with neural connections 1450. The layers 1460 and 1480 are connected with neural connections 1470. Data flows forward via inputs 1412, 1414, 1416 from the input layer 1420 to the output layer 1480 and to an output 1490.

The layer 1420 is an input layer that, in the example of FIG. 14, includes a plurality of nodes 1422, 1424, 1426. The layers 1440 and 1460 are hidden layers and include, the example of FIG. 14, nodes 1442, 1444, 1446, 1448, 1462, 1464, 1466, 1468. The neural network 1400 may include more or less hidden layers 1440 and 1460 than shown. The layer 1480 is an output layer and includes, in the example of FIG. 14, a node 1482 with an output 1490. Each input 1412-1416 corresponds to a node 1422-1426 of the input layer 1420, and each node 1422-1426 of the input layer 1420 has a connection 1430 to each node 1442-1448 of the hidden layer 1440. Each node 1442-1448 of the hidden layer 1440 has a connection 1450 to each node 1462-1468 of the hidden layer 1460. Each node 1462-1468 of the hidden layer 1460 has a connection 1470 to the output layer 1480. The output layer 1480 has an output 1490 to provide an output from the example neural network 1400.

Of connections 1430, 1450, and 1470 certain example connections 1432, 1452, 1472 may be given added weight while other example connections 1434, 1454, 1474 may be given less weight in the neural network 1400. Input nodes 1422-1426 are activated through receipt of input data via inputs 1412-1416, for example. Nodes 1442-1448 and 1462-1468 of hidden layers 1440 and 1460 are activated through the forward flow of data through the network 1400 via the connections 1430 and 1450, respectively. Node 1482 of the output layer 1480 is activated after data processed in hidden layers 1440 and 1460 is sent via connections 1470. When the output node 1482 of the output layer 1480 is activated, the node 1482 outputs an appropriate value based on processing accomplished in hidden layers 1440 and 1460 of the neural network 1400.

Example Healthcare Systems and Environments

Health information, also referred to as healthcare information and/or healthcare data, relates to information generated and/or used by a healthcare entity. Health information can be information associated with health of one or more patients, for example. Health information may include protected health information (PHI), as outlined in the Health Insurance Portability and Accountability Act (HIPAA), which is identifiable as associated with a particular patient and is protected from unauthorized disclosure. Health information can be organized as internal information and external information. Internal information includes patient encounter information (e.g., patient-specific data, aggregate data, comparative data, etc.) and general healthcare operations information, etc. External information includes comparative data, expert and/or knowledge-based data, etc. Information can have both a clinical (e.g., diagnosis, treatment, prevention, etc.) and administrative (e.g., scheduling, billing, management, etc.) purpose.

Institutions, such as healthcare institutions, having complex network support environments and sometimes chaotically driven process flows utilize secure handling and safeguarding of the flow of sensitive information (e.g., personal privacy). A need for secure handling and safeguarding of information increases as a demand for flexibility, volume, and speed of exchange of such information grows. For example, healthcare institutions provide enhanced control and safeguarding of the exchange and storage of sensitive patient protected health information (PHI) between diverse locations to improve hospital operational efficiency in an operational environment typically having a chaotic-driven demand by patients for hospital services. In certain examples, patient identifying information can be masked or even stripped from certain data depending upon where the data is stored and who has access to that data. In some examples, PHI that has been “de-identified” can be re-identified based on a key and/or other encoder/decoder.

A healthcare information technology infrastructure can be adapted to service multiple business interests while providing clinical information and services. Such an infrastructure may include a centralized capability including, for example, a data repository, reporting, discrete data exchange/connectivity, “smart” algorithms, personalization/consumer decision support, etc. This centralized capability provides information and functionality to a plurality of users including medical devices, electronic records, access portals, pay for performance (P4P), chronic disease models, and clinical health information exchange/regional health information organization (HIE/RHIO), and/or enterprise pharmaceutical studies, home health, for example.

Interconnection of multiple data sources helps enable an engagement of all relevant members of a patient's care team and helps improve an administrative and management burden on the patient for managing his or her care. Particularly, interconnecting the patient's electronic medical record and/or other medical data can help improve patient care and management of patient information. Furthermore, patient care compliance, including surgical procedure and/or other protocol compliance, is facilitated by providing tools that automatically adapt to the specific and changing health conditions of the patient and provide comprehensive education and compliance tools for practitioner and/or patient to drive positive health outcomes.

In certain examples, healthcare information can be distributed among multiple applications using a variety of database and storage technologies and data formats. To provide a common interface and access to data residing across these applications, a connectivity framework (CF) can be provided which leverages common data and service models (CDM and CSM) and service oriented technologies, such as an enterprise service bus (ESB) to provide access to the data.

In certain examples, a variety of user interface frameworks and technologies can be used to build applications for health information systems including, but not limited to, MICROSOFT® ASP.NET, AJAX®, MICROSOFT® Windows Presentation Foundation, GOOGLE® Web Toolkit, MICROSOFT® Silverlight, ADOBE®, and others. Applications can be composed from libraries of information widgets to display multi-content and multi-media information, for example. In addition, the framework enables users to tailor layout of applications and interact with underlying data.

In certain examples, an advanced Service-Oriented Architecture (SOA) with a modern technology stack helps provide robust interoperability, reliability, and performance. Example SOA includes a three-fold interoperability strategy including a central repository (e.g., a central repository built from Health Level Seven (HL7) transactions), services for working in federated environments, and visual integration with third-party applications. Certain examples provide portable content enabling plug 'n play content exchange among healthcare organizations. A standardized vocabulary using common standards (e.g., LOINC, SNOMED CT, RxNorm, FDB, ICD-9, ICD-10, CCDA, etc.) is used for interoperability, for example. Certain examples provide an intuitive user interface to help minimize end-user training. Certain examples facilitate user-initiated launching of third-party applications directly from a desktop interface to help provide a seamless workflow by sharing user, patient, and/or other contexts. Certain examples provide real-time (or at least substantially real time assuming some system delay) patient data from one or more information technology (IT) systems and facilitate comparison(s) against evidence-based best practices. Certain examples provide one or more dashboards for specific sets of patients and/or practitioners, such as surgeons, surgical technicians, nurses, assistants, radiologists, administrators, etc. Dashboard(s) can be based on condition, role, and/or other criteria to indicate variation(s) from a desired practice, for example.

Example Healthcare Information System

An information system can be defined as an arrangement of information/data, processes, and information technology that interact to collect, process, store, and provide informational output to support delivery of healthcare to one or more patients. Information technology includes computer technology (e.g., hardware and software) along with data and telecommunications technology (e.g., data, image, and/or voice network, etc.).

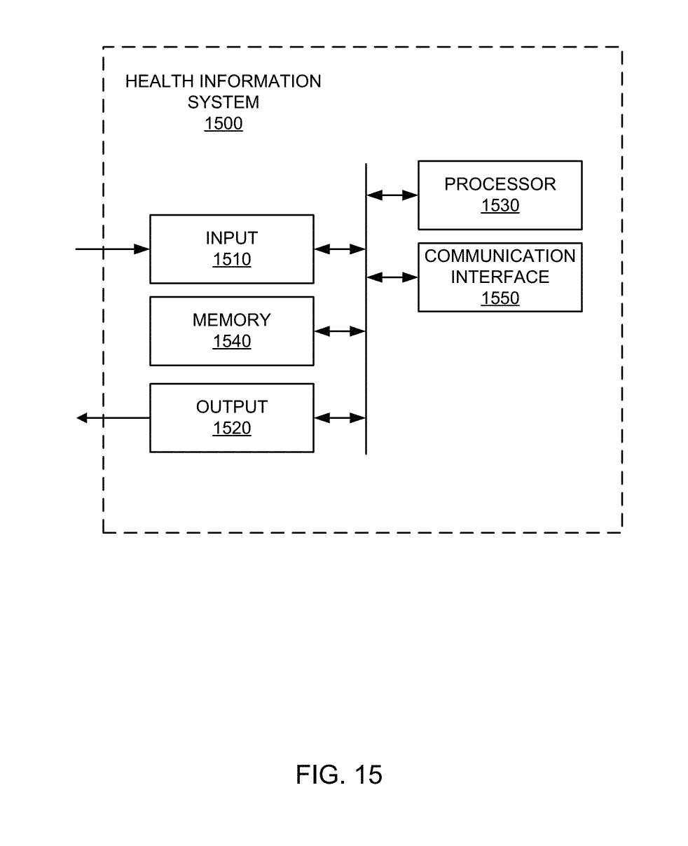

Turning now to the figures, FIG. 15 shows a block diagram of an example healthcare-focused information system 1500. Example system 1500 can be configured to implement a variety of systems (e.g., scheduler, care system, care ecosystem, monitoring system, portal, services, supporting functionality, digital twin 130, etc.) and processes including image storage (e.g., picture archiving and communication system (PACS), etc.), image processing and/or analysis, radiology reporting and/or review (e.g., radiology information system (RIS), etc.), computerized provider order entry (CPOE) system, clinical decision support, patient monitoring, population health management (e.g., population health management system (PHMS), health information exchange (HIE), etc.), healthcare data analytics, cloud-based image sharing, electronic medical record (e.g., electronic medical record system (EMR), electronic health record system (EHR), electronic patient record (EPR), personal health record system (PHR), etc.), and/or other health information system (e.g., clinical information system (CIS), hospital information system (HIS), patient data management system (PDMS), laboratory information system (LIS), cardiovascular information system (CVIS), etc.

As illustrated in FIG. 15, the example information system 1500 includes an input 1510, an output 1520, a processor 1530, a memory 1540, and a communication interface 1550. The components of example system 1500 can be integrated in one device or distributed over two or more devices.

Example input 1510 may include a keyboard, a touch-screen, a mouse, a trackball, a track pad, optical barcode recognition, voice command, etc. or combination thereof used to communicate an instruction or data to system 1500. Example input 1510 may include an interface between systems, between user(s) and system 1500, etc.

Example output 1520 can provide a display generated by processor 1530 for visual illustration on a monitor or the like. The display can be in the form of a network interface or graphic user interface (GUI) to exchange data, instructions, or illustrations on a computing device via communication interface 1550, for example. Example output 1520 may include a monitor (e.g., liquid crystal display (LCD), plasma display, cathode ray tube (CRT), etc.), light emitting diodes (LEDs), a touch-screen, a printer, a speaker, or other conventional display device or combination thereof.

Example processor 1530 includes hardware and/or software configuring the hardware to execute one or more tasks and/or implement a particular system configuration. Example processor 1530 processes data received at input 1510 and generates a result that can be provided to one or more of output 1520, memory 1540, and communication interface 1550. For example, example processor 1530 can take object detection information provided by the sensor 310, 735 via input 1510 with respect to items in the surgical field 520 and can generate a report and/or other guidance regarding the items and protocol adherence via the output 1520. As another example, processor 1530 can process imaging protocol information obtained via input 1510 to provide an updated configuration for an imaging scanner via communication interface 1550.

Example memory 1540 can include a relational database, an object-oriented database, a Hadoop data construct repository, a data dictionary, a clinical data repository, a data warehouse, a data mart, a vendor neutral archive, an enterprise archive, etc. Example memory 1540 stores images, patient data, best practices, clinical knowledge, analytics, reports, etc. Example memory 1540 can store data and/or instructions for access by the processor 1530 (e.g., including the digital twin 130). In certain examples, memory 1540 can be accessible by an external system via the communication interface 1550.

Example communication interface 1550 facilitates transmission of electronic data within and/or among one or more systems. Communication via communication interface 1550 can be implemented using one or more protocols. In some examples, communication via communication interface 1550 occurs according to one or more standards (e.g., Digital Imaging and Communications in Medicine (DICOM), Health Level Seven (HL7), ANSI X12N, etc.), or proprietary systems. Example communication interface 1550 can be a wired interface (e.g., a data bus, a Universal Serial Bus (USB) connection, etc.) and/or a wireless interface (e.g., radio frequency, infrared (IR), near field communication (NFC), etc.). For example, communication interface 1550 may communicate via wired local area network (LAN), wireless LAN, wide area network (WAN), etc. using any past, present, or future communication protocol (e.g., BLUETOOTH™, USB 2.0, USB 3.0, etc.).

In certain examples, a Web-based portal or application programming interface (API), may be used to facilitate access to information, protocol library, imaging system configuration, patient care and/or practice management, etc. Information and/or functionality available via the Web-based portal may include one or more of order entry, laboratory test results review system, patient information, clinical decision support, medication management, scheduling, electronic mail and/or messaging, medical resources, etc. In certain examples, a browser-based interface can serve as a zero footprint, zero download, and/or other universal viewer for a client device.

In certain examples, the Web-based portal or API serves as a central interface to access information and applications, for example. Data may be viewed through the Web-based portal or viewer, for example. Additionally, data may be manipulated and propagated using the Web-based portal, for example. Data may be generated, modified, stored and/or used and then communicated to another application or system to be modified, stored and/or used, for example, via the Web-based portal, for example.

The Web-based portal or API may be accessible locally (e.g., in an office) and/or remotely (e.g., via the Internet and/or other private network or connection), for example. The Web-based portal may be configured to help or guide a user in accessing data and/or functions to facilitate patient care and practice management, for example. In certain examples, the Web-based portal may be configured according to certain rules, preferences and/or functions, for example. For example, a user may customize the Web portal according to particular desires, preferences and/or requirements.

Example Healthcare Infrastructure

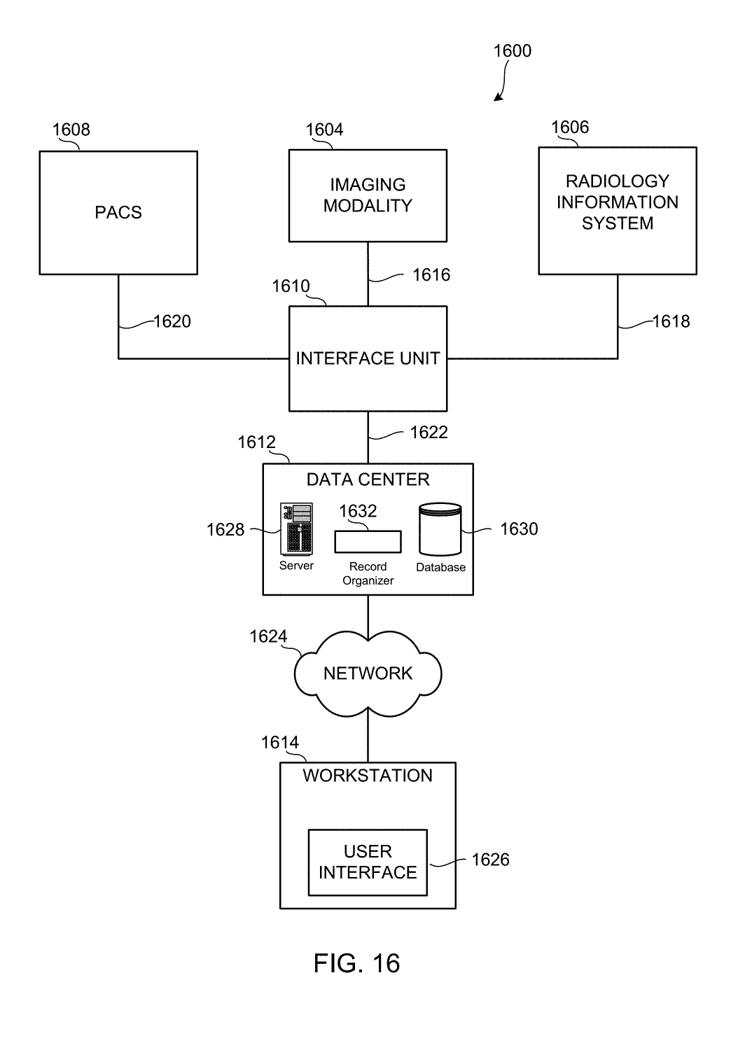

FIG. 16 shows a block diagram of an example healthcare information infrastructure 1600 including one or more subsystems (e.g., scheduler, care system, care ecosystem, monitoring system, portal, services, supporting functionality, digital twin 130, etc.) such as the example healthcare-related information system 1500 illustrated in FIG. 15. Example healthcare system 1600 includes an imaging modality 1604, a RIS 1606, a PACS 1608, an interface unit 1610, a data center 1612, and a workstation 1614. In the illustrated example, scanner/modality 1604, RIS 1606, and PACS 1608 are housed in a healthcare facility and locally archived. However, in other implementations, imaging modality 1604, RIS 1606, and/or PACS 1608 may be housed within one or more other suitable locations. In certain implementations, one or more of PACS 1608, RIS 1606, modality 1604, etc., may be implemented remotely via a thin client and/or downloadable software solution. Furthermore, one or more components of the healthcare system 1600 can be combined and/or implemented together. For example, RIS 1606 and/or PACS 1608 can be integrated with the imaging scanner 1604; PACS 1608 can be integrated with RIS 1606; and/or the three example systems 1604, 1606, and/or 1608 can be integrated together. In other example implementations, healthcare system 1600 includes a subset of the illustrated systems 1604, 1606, and/or 1608. For example, healthcare system 1600 may include only one or two of the modality 1604, RIS 1606, and/or PACS 1608. Information (e.g., scheduling, test results, exam image data, observations, diagnosis, etc.) can be entered into the scanner 1604, RIS 1606, and/or PACS 1608 by healthcare practitioners (e.g., radiologists, physicians, and/or technicians) and/or administrators before and/or after patient examination. One or more of the imaging scanner 1604, RIS 1606, and/or PACS 1608 can communicate with equipment and system(s) in an operating room, patient room, etc., to track activity, correlate information, generate reports and/or next actions, and the like.

The RIS 1606 stores information such as, for example, radiology reports, radiology exam image data, messages, warnings, alerts, patient scheduling information, patient demographic data, patient tracking information, and/or physician and patient status monitors. Additionally, RIS 1606 enables exam order entry (e.g., ordering an x-ray of a patient) and image and film tracking (e.g., tracking identities of one or more people that have checked out a film). In some examples, information in RIS 1606 is formatted according to the HL-7 (Health Level Seven) clinical communication protocol. In certain examples, a medical exam distributor is located in RIS 1606 to facilitate distribution of radiology exams to a radiologist workload for review and management of the exam distribution by, for example, an administrator.

PACS 1608 stores medical images (e.g., x-rays, scans, three-dimensional renderings, etc.) as, for example, digital images in a database or registry. In some examples, the medical images are stored in PACS 1608 using the Digital Imaging and Communications in Medicine (DICOM) format. Images are stored in PACS 1608 by healthcare practitioners (e.g., imaging technicians, physicians, radiologists) after a medical imaging of a patient and/or are automatically transmitted from medical imaging devices to PACS 1608 for storage. In some examples, PACS 1608 can also include a display device and/or viewing workstation to enable a healthcare practitioner or provider to communicate with PACS 1608.

The interface unit 1610 includes a hospital information system interface connection 1616, a radiology information system interface connection 1618, a PACS interface connection 1620, and a data center interface connection 1622. Interface unit 1610 facilities communication among imaging modality 1604, RIS 1606, PACS 1608, and/or data center 1612. Interface connections 1616, 1618, 1620, and 1622 can be implemented by, for example, a Wide Area Network (WAN) such as a private network or the Internet. Accordingly, interface unit 1610 includes one or more communication components such as, for example, an Ethernet device, an asynchronous transfer mode (ATM) device, an 802.11 device, a DSL modem, a cable modem, a cellular modem, etc. In turn, the data center 1612 communicates with workstation 1614, via a network 1624, implemented at a plurality of locations (e.g., a hospital, clinic, doctor's office, other medical office, or terminal, etc.). Network 1624 is implemented by, for example, the Internet, an intranet, a private network, a wired or wireless Local Area Network, and/or a wired or wireless Wide Area Network. In some examples, interface unit 1610 also includes a broker (e.g., a Mitra Imaging's PACS Broker) to allow medical information and medical images to be transmitted together and stored together.

Interface unit 1610 receives images, medical reports, administrative information, exam workload distribution information, surgery and/or other protocol information, and/or other clinical information from the information systems 1604, 1606, 1608 via the interface connections 1616, 1618, 1620. If necessary (e.g., when different formats of the received information are incompatible), interface unit 1610 translates or reformats (e.g., into Structured Query Language (“SQL”) or standard text) the medical information, such as medical reports, to be properly stored at data center 1612. The reformatted medical information can be transmitted using a transmission protocol to enable different medical information to share common identification elements, such as a patient name or social security number. Next, interface unit 1610 transmits the medical information to data center 1612 via data center interface connection 1622. Finally, medical information is stored in data center 1612 in, for example, the DICOM format, which enables medical images and corresponding medical information to be transmitted and stored together.

The medical information is later viewable and easily retrievable at workstation 1614 (e.g., by their common identification element, such as a patient name or record number). Workstation 1614 can be any equipment (e.g., a personal computer) capable of executing software that permits electronic data (e.g., medical reports) and/or electronic medical images (e.g., x-rays, ultrasounds, MRI scans, etc.) to be acquired, stored, or transmitted for viewing and operation. Workstation 1614 receives commands and/or other input from a user via, for example, a keyboard, mouse, track ball, microphone, etc. Workstation 1614 can implement a user interface 1626 to enable a healthcare practitioner and/or administrator to interact with healthcare system 1600. For example, in response to a request from a physician, user interface 1626 presents a patient medical history, preference card, surgical protocol list, etc. In other examples, a radiologist is able to retrieve and manage a workload of exams distributed for review to the radiologist via user interface 1626. In further examples, an administrator reviews radiologist workloads, exam allocation, and/or operational statistics associated with the distribution of exams via user interface 1626. In some examples, the administrator adjusts one or more settings or outcomes via user interface 1626. In some examples, a surgeon and/or supporting nurses, technicians, etc., review a surgical preference card and protocol information in preparation for, during, and/or after a surgical procedure.

Example data center 1612 of FIG. 16 is an archive to store information such as images, data, medical reports, patient medical records, preference cards, etc. In addition, data center 1612 can also serve as a central conduit to information located at other sources such as, for example, local archives, hospital information systems/radiology information systems (e.g., HIS 1604 and/or RIS 1606), or medical imaging/storage systems (e.g., PACS 1608 and/or connected imaging modalities). That is, the data center 1612 can store links or indicators (e.g., identification numbers, patient names, or record numbers) to information. In the illustrated example, data center 1612 is managed by an application server provider (ASP) and is located in a centralized location that can be accessed by a plurality of systems and facilities (e.g., hospitals, clinics, doctor's offices, other medical offices, and/or terminals). In some examples, data center 1612 can be spatially distant from the imaging modality 1604, RIS 1606, and/or PACS 1608. In certain examples, the data center 1612 can be located in and/or near the cloud (e.g., on a cloud-based server, an edge device, etc.).

Example data center 1612 of FIG. 16 includes a server 1628, a database 1630, and a record organizer 1632. Server 1628 receives, processes, and conveys information to and from the components of healthcare system 1600. Database 1630 stores the medical information described herein and provides access thereto. Example record organizer 1632 of FIG. 16 manages patient medical histories, for example. Record organizer 1632 can also assist in procedure scheduling, protocol adherence, procedure follow-up, etc.

Certain examples can be implemented as cloud-based clinical information systems and associated methods of use. An example cloud-based clinical information system enables healthcare entities (e.g., patients, clinicians, sites, groups, communities, and/or other entities) to share information via web-based applications, cloud storage and cloud services. For example, the cloud-based clinical information system may enable a first clinician to securely upload information into the cloud-based clinical information system to allow a second clinician to view and/or download the information via a web application. Thus, for example, the first clinician may upload an x-ray imaging protocol, surgical procedure protocol, etc., into the cloud-based clinical information system, and the second clinician may view and download the x-ray imaging protocol, surgical procedure protocol, etc., via a web browser and/or download the x-ray imaging protocol, surgical procedure protocol, etc., onto a local information system employed by the second clinician.

In certain examples, users (e.g., a patient and/or care provider) can access functionality provided by system 1600 via a software-as-a-service (SaaS) implementation over a cloud or other computer network, for example. In certain examples, all or part of system 1600 can also be provided via platform as a service (PaaS), infrastructure as a service (IaaS), etc. For example, system 1600 can be implemented as a cloud-delivered Mobile Computing Integration Platform as a Service. A set of Web-based, mobile, and/or other applications enable users to interact with the PaaS, for example.

Industrial Internet Examples

The Internet of things (also referred to as the “Industrial Internet”) relates to an interconnection between a device that can use an Internet connection to talk with other devices and/or applications on the network. Using the connection, devices can communicate to trigger events/actions (e.g., changing temperature, turning on/off, providing a status, etc.). In certain examples, machines can be merged with “big data” to improve efficiency and operations, provide improved data mining, facilitate better operation, etc.

Big data can refer to a collection of data so large and complex that it becomes difficult to process using traditional data processing tools/methods. Challenges associated with a large data set include data capture, sorting, storage, search, transfer, analysis, and visualization. A trend toward larger data sets is due at least in part to additional information derivable from analysis of a single large set of data, rather than analysis of a plurality of separate, smaller data sets. By analyzing a single large data set, correlations can be found in the data, and data quality can be evaluated.

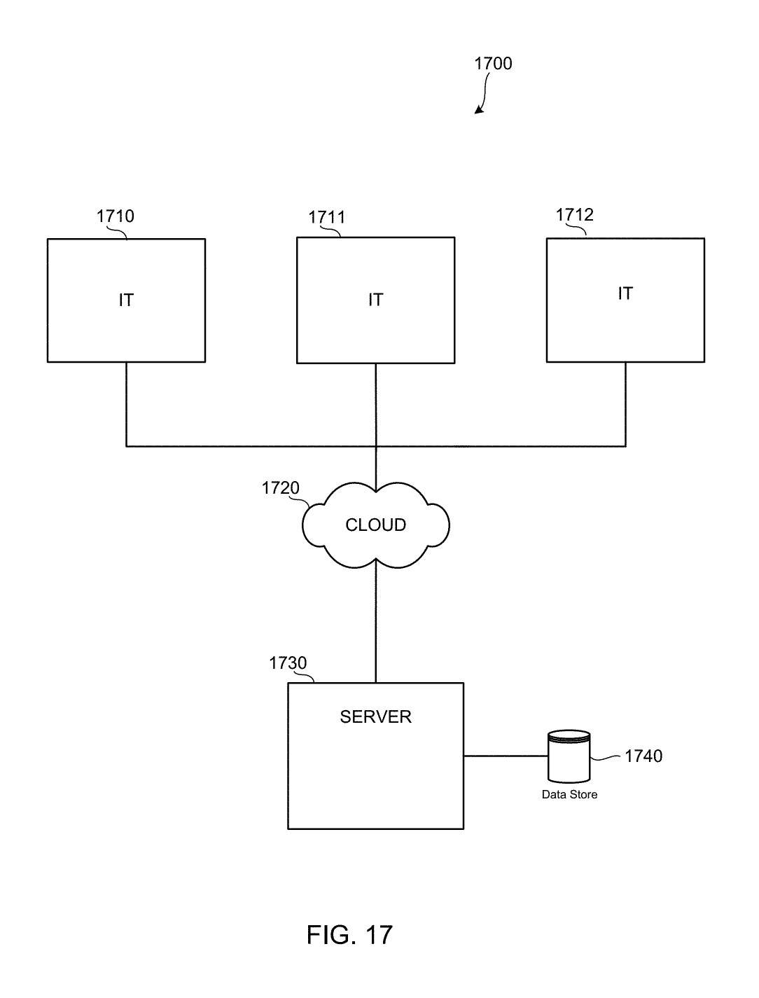

FIG. 17 illustrates an example industrial internet configuration 1700. Example configuration 1700 includes a plurality of health-focused systems 1710-1712, such as a plurality of health information systems 1500 (e.g., PACS, RIS, EMR, PHMS and/or other scheduler, care system, care ecosystem, monitoring system, services, supporting functionality, digital twin 130, etc.) communicating via industrial internet infrastructure 1700. Example industrial internet 1700 includes a plurality of health-related information systems 1710-1712 communicating via a cloud 1720 with a server 1730 and associated data store 1740.

As shown in the example of FIG. 17, a plurality of devices (e.g., information systems, imaging modalities, etc.) 1710-1712 can access a cloud 1720, which connects the devices 1710-1712 with a server 1730 and associated data store 1740. Information systems, for example, include communication interfaces to exchange information with server 1730 and data store 1740 via the cloud 1720. Other devices, such as medical imaging scanners, patient monitors, object scanners, location trackers, etc., can be outfitted with sensors and communication interfaces to enable them to communicate with each other and with the server 1730 via the cloud 1720.

Thus, machines 1710-1712 within system 1700 become “intelligent” as a network with advanced sensors, controls, analytical based decision support and hosting software applications. Using such an infrastructure, advanced analytics can be provided to associated data. The analytics combines physics-based analytics, predictive algorithms, automation, and deep domain expertise. Via cloud 1720, devices 1710-1712 and associated people can be connected to support more intelligent design, operations, maintenance, and higher server quality and safety, for example.

Using the industrial internet infrastructure, for example, a proprietary machine data stream can be extracted from a device 1710. Machine-based algorithms and data analysis are applied to the extracted data. Data visualization can be remote, centralized, etc. Data is then shared with authorized users, and any gathered and/or gleaned intelligence is fed back into the machines 1710-1712.

While progress with industrial equipment automation has been made over the last several decades, and assets have become ‘smarter,’ the intelligence of any individual asset pales in comparison to intelligence that can be gained when multiple smart devices are connected together. Aggregating data collected from or about multiple assets can enable users to improve business processes, for example by improving effectiveness of asset maintenance or improving operational performance if appropriate industrial-specific data collection and modeling technology is developed and applied.

In an example, data from one or more sensors can be recorded or transmitted to a cloud-based or other remote computing environment. Insights gained through analysis of such data in a cloud-based computing environment can lead to enhanced asset designs, or to enhanced software algorithms for operating the same or similar asset at its edge, that is, at the extremes of its expected or available operating conditions. For example, sensors associated with the surgical field 502 can supplement the modeled information of the digital twin 130, which can be stored and/or otherwise instantiated in a cloud-based computing environment for access by a plurality of systems with respect to a healthcare procedure and/or protocol.

Systems and methods described herein can include using a “cloud” or remote or distributed computing resource or service. The cloud can be used to receive, relay, transmit, store, analyze, or otherwise process information for or about the digital twin 130, for example. In an example, a cloud computing system includes at least one processor circuit, at least one database, and a plurality of users or assets that are in data communication with the cloud computing system. The cloud computing system can further include or can be coupled with one or more other processor circuits or modules configured to perform a specific task, such as to perform tasks related to patient monitoring, diagnosis, treatment (e.g., surgical procedure, etc.), scheduling, etc., via the digital twin 130.

Data Mining Examples

Imaging informatics includes determining how to tag and index a large amount of data acquired in diagnostic imaging in a logical, structured, and machine-readable format. By structuring data logically, information can be discovered and utilized by algorithms that represent clinical pathways and decision support systems. Data mining can be used to help ensure patient safety, reduce disparity in treatment, provide clinical decision support, etc. Mining both structured and unstructured data from radiology reports, as well as actual image pixel data, can be used to tag and index both imaging reports and the associated images themselves. Data mining can be used to provide information to the digital twin 130, for example.

Example Methods of Use

Clinical workflows are typically defined to include one or more steps or actions to be taken in response to one or more events and/or according to a schedule. Events may include receiving a healthcare message associated with one or more aspects of a clinical record, opening a record(s) for new patient(s), receiving a transferred patient, reviewing and reporting on an image, executing orders for specific care, signing off on orders for a discharge, and/or any other instance and/or situation that requires or dictates responsive action or processing. The actions or steps of a clinical workflow may include placing an order for one or more clinical tests, scheduling a procedure, requesting certain information to supplement a received healthcare record, retrieving additional information associated with a patient, providing instructions to a patient and/or a healthcare practitioner associated with the treatment of the patient, conducting and/or facilitating conduct of a procedure and/or other clinical protocol, radiology image reading, dispatching room cleaning and/or patient transport, and/or any other action useful in processing healthcare information or causing critical path care activities to progress. The defined clinical workflows may include manual actions or steps to be taken by, for example, an administrator or practitioner, electronic actions or steps to be taken by a system or device, and/or a combination of manual and electronic action(s) or step(s). While one entity of a healthcare enterprise may define a clinical workflow for a certain event in a first manner, a second entity of the healthcare enterprise may define a clinical workflow of that event in a second, different manner. In other words, different healthcare entities may treat or respond to the same event or circumstance in different fashions. Differences in workflow approaches may arise from varying preferences, capabilities, requirements or obligations, standards, protocols, etc. among the different healthcare entities.

In certain examples, a medical exam conducted on a patient can involve review by a healthcare practitioner, such as a radiologist, to obtain, for example, diagnostic information from the exam. In a hospital setting, medical exams can be ordered for a plurality of patients, all of which require review by an examining practitioner. Each exam has associated attributes, such as a modality, a part of the human body under exam, and/or an exam priority level related to a patient criticality level. Hospital administrators, in managing distribution of exams for review by practitioners, can consider the exam attributes as well as staff availability, staff credentials, and/or institutional factors such as service level agreements and/or overhead costs.

Additional workflows can be facilitated such as bill processing, revenue cycle mgmt., population health management, patient identity, consent management, etc.

While example implementations are illustrated in conjunction with FIGS. 1-17, elements, processes and/or devices illustrated in conjunction with FIGS. 1-17 may be combined, divided, re-arranged, omitted, eliminated and/or implemented in any other way. Further, components disclosed and described herein can be implemented by hardware, machine readable instructions, software, firmware and/or any combination of hardware, machine readable instructions, software and/or firmware. Thus, for example, components disclosed and described herein can be implemented by analog and/or digital circuit(s), logic circuit(s), programmable processor(s), application specific integrated circuit(s) (ASIC(s)), programmable logic device(s) (PLD(s)) and/or field programmable logic device(s) (FPLD(s)). When reading any of the apparatus or system claims of this patent to cover a purely software and/or firmware implementation, at least one of the components is/are hereby expressly defined to include a tangible computer readable storage device or storage disk such as a memory, a digital versatile disk (DVD), a compact disk (CD), a Blu-ray disk, etc. storing the software and/or firmware.

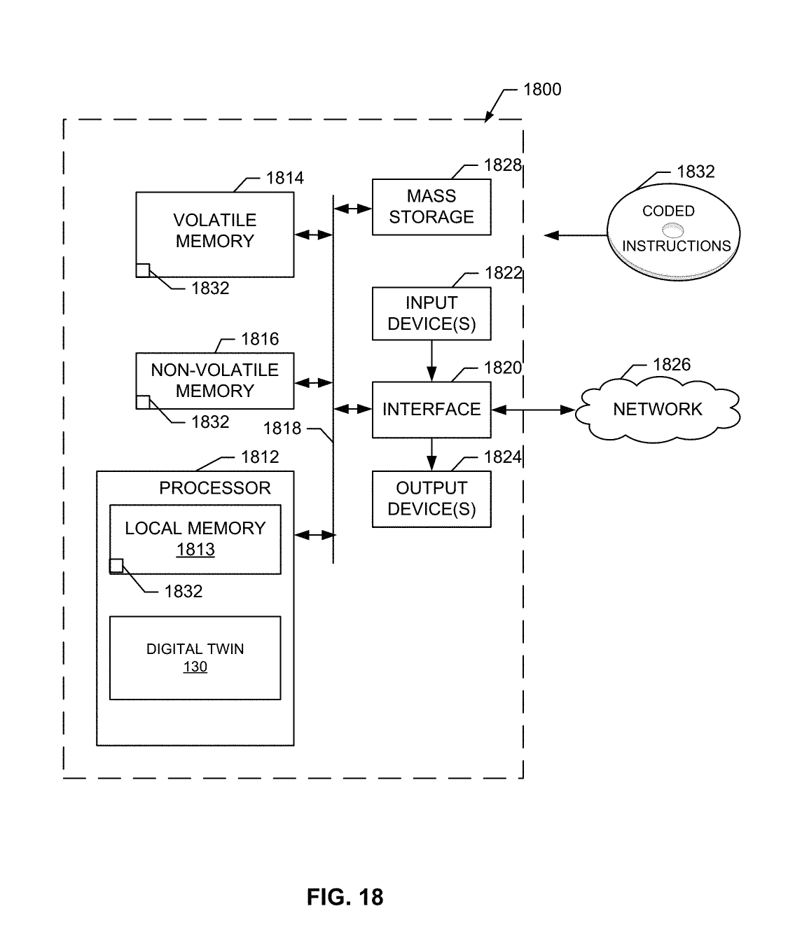

Flowcharts representative of example machine readable instructions for implementing components disclosed and described herein are shown in conjunction with FIGS. 9, 11, and 13. In the examples, the machine readable instructions include a program for execution by a processor such as the processor 1812 shown in the example processor platform 1800 discussed below in connection with FIG. 18. The program may be embodied in machine readable instructions stored on a tangible computer readable storage medium such as a CD-ROM, a floppy disk, a hard drive, a digital versatile disk (DVD), a Blu-ray disk, or a memory associated with the processor 1812, but the entire program and/or parts thereof could alternatively be executed by a device other than the processor 1812 and/or embodied in firmware or dedicated hardware. Further, although the example program is described with reference to the flowcharts illustrated in conjunction with at least FIGS. 9, 11, and 13, many other methods of implementing the components disclosed and described herein may alternatively be used. For example, the order of execution of the blocks may be changed, and/or some of the blocks described may be changed, eliminated, or combined. Although the flowcharts of at least FIGS. 9, 11, and 13 depict example operations in an illustrated order, these operations are not exhaustive and are not limited to the illustrated order. In addition, various changes and modifications may be made by one skilled in the art within the spirit and scope of the disclosure. For example, blocks illustrated in the flowchart may be performed in an alternative order or may be performed in parallel.

As mentioned above, the example components, data structures, and/or processes of at least FIGS. 1-17 can be implemented using coded instructions (e.g., computer and/or machine readable instructions) stored on a tangible computer readable storage medium such as a hard disk drive, a flash memory, a read-only memory (ROM), a compact disk (CD), a digital versatile disk (DVD), a cache, a random-access memory (RAM) and/or any other storage device or storage disk in which information is stored for any duration (e.g., for extended time periods, permanently, for brief instances, for temporarily buffering, and/or for caching of the information). As used herein, the term tangible computer readable storage medium is expressly defined to include any type of computer readable storage device and/or storage disk and to exclude propagating signals and to exclude transmission media. As used herein, “tangible computer readable storage medium” and “tangible machine readable storage medium” are used interchangeably. Additionally or alternatively, the example components, data structures, and/or processes of at least FIGS. 1-17 can be implemented using coded instructions (e.g., computer and/or machine readable instructions) stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media. As used herein, when the phrase “at least” is used as the transition term in a preamble of a claim, it is open-ended in the same manner as the term “comprising” is open ended. In addition, the term “including” is open-ended in the same manner as the term “comprising” is open-ended.

FIG. 18 is a block diagram of an example processor platform 1800 structured to executing the instructions of at least FIGS. 9 and 11-17 to implement the example components disclosed and described herein. The processor platform 1800 can be, for example, a server, a personal computer, a mobile device (e.g., a cell phone, a smart phone, a tablet such as an iPad™), a personal digital assistant (PDA), an Internet appliance, or any other type of computing device.

The processor platform 1800 of the illustrated example includes a processor 1812. The processor 1812 of the illustrated example is hardware. For example, the processor 1812 can be implemented by integrated circuits, logic circuits, microprocessors or controllers from any desired family or manufacturer.

The processor 1812 of the illustrated example includes a local memory 1813 (e.g., a cache). The example processor 1812 of FIG. 18 executes the instructions of at least FIGS. 9, 11 and 13 to implement the digital twin 130 and associated components such as the processor 710, memory 720, input 730, output 740, etc. The processor 1812 of the illustrated example is in communication with a main memory including a volatile memory 1814 and a non-volatile memory 1816 via a bus 1818. The volatile memory 1814 may be implemented by Synchronous Dynamic Random Access Memory (SDRAM), Dynamic Random Access Memory (DRAM), RAMBUS Dynamic Random Access Memory (RDRAM) and/or any other type of random access memory device. The non-volatile memory 1816 may be implemented by flash memory and/or any other desired type of memory device. Access to the main memory 1814, 1816 is controlled by a clock controller.

The processor platform 1800 of the illustrated example also includes an interface circuit 1820. The interface circuit 1820 may be implemented by any type of interface standard, such as an Ethernet interface, a universal serial bus (USB), and/or a PCI express interface.

In the illustrated example of FIG. 18, one or more input devices 1822 are connected to the interface circuit 1820. The input device(s) 1822 permit(s) a user to enter data and commands into the processor 1812. The input device(s) can be implemented by, for example, a sensor, a microphone, a camera (still or video), a keyboard, a button, a mouse, a touchscreen, a track-pad, a trackball, isopoint and/or a voice recognition system.

One or more output devices 1824 are also connected to the interface circuit 1820 of the illustrated example. The output devices 1824 can be implemented, for example, by display devices (e.g., a light emitting diode (LED), an organic light emitting diode (OLED), a liquid crystal display, a cathode ray tube display (CRT), a touchscreen, a tactile output device, and/or speakers). The interface circuit 1820 of the illustrated example, thus, typically includes a graphics driver card, a graphics driver chip or a graphics driver processor.

The interface circuit 1820 of the illustrated example also includes a communication device such as a transmitter, a receiver, a transceiver, a modem and/or network interface card to facilitate exchange of data with external machines (e.g., computing devices of any kind) via a network 1826 (e.g., an Ethernet connection, a digital subscriber line (DSL), a telephone line, coaxial cable, a cellular telephone system, etc.).

The processor platform 1800 of the illustrated example also includes one or more mass storage devices 1828 for storing software and/or data. Examples of such mass storage devices 1828 include floppy disk drives, hard drive disks, compact disk drives, Blu-ray disk drives, RAID systems, and digital versatile disk (DVD) drives.

The coded instructions 1832 of FIG. 18 may be stored in the mass storage device 1828, in the volatile memory 1814, in the non-volatile memory 1816, and/or on a removable tangible computer readable storage medium such as a CD or DVD.

From the foregoing, it will be appreciated that the above disclosed methods, apparatus, and articles of manufacture have been disclosed to create and dynamically update a patient digital twin that can be used in patient simulation, analysis, diagnosis, and treatment to improve patient health outcome.

Although certain example methods, apparatus and articles of manufacture have been described herein, the scope of coverage of this patent is not limited thereto. On the contrary, this patent covers all methods, apparatus and articles of manufacture fairly falling within the scope of the claims of this patent.

What is claimed is:

1. An apparatus comprising:
a processor and a memory, the processor to configure the memory according to a digital twin of a first healthcare procedure, the digital twin including a data structure created from tasks defining the first healthcare procedure and a list of items to be used in the first healthcare procedure to model the tasks of the first healthcare procedure and items associated with each task of the first healthcare procedure, the digital twin arranged for query and simulation via the processor to model the first healthcare procedure for a first patient,
the digital twin to at least:
receive input regarding a first item at a first location;
compare the first item to the items associated with each task of the first healthcare procedure; and
when the first item matches an item associated with a task of the first healthcare procedure, record the first item and approval for the first healthcare procedure and update the digital twin based on the first item; and
when the first item does not match an item associated with a task of the first healthcare procedure, log the first item.

2. The apparatus of claim 1, further including a sensor to identify the first item at the first location.

3. The apparatus of claim 2, wherein the sensor is to verify whether the first item was used in the first healthcare procedure for the first patient.

4. The apparatus of claim 3, wherein, when the first item was used in the first healthcare procedure for the first patient, a preference card is updated based on the first item.

5. The apparatus of claim 2, wherein the sensor is incorporated into at least one of glasses or an eye shield, and wherein information regarding the first item is displayed via the at least one of glasses or eye shield.

6. The apparatus of claim 2, wherein the sensor is incorporated into a cart with a computing device.

7. The apparatus of claim 1, wherein the digital twin is periodically retrained and redeployed based on feedback including at least one of the update or the log.

8. A computer-readable storage medium comprising instructions which, when executed by a processor, cause a machine to implement at least:
a digital twin of a first healthcare procedure, the digital twin including a data structure created from tasks defining the first healthcare procedure and a list of items to be used in the first healthcare procedure to model the tasks of the first healthcare procedure and items associated with each task of the first healthcare procedure, the digital twin arranged for query and simulation via the processor to model the first healthcare procedure for a first patient,
the digital twin to at least:
receive input regarding a first item at a first location;
compare the first item to the items associated with each task of the first healthcare procedure; and
when the first item matches an item associated with a task of the first healthcare procedure, record the first item and approval for the first healthcare procedure and update the digital twin based on the first item; and
when the first item does not match an item associated with a task of the first healthcare procedure, log the first item.

9. The computer-readable storage medium of claim 8, wherein the digital twin is to interact with a sensor to identify the first item at the first location.

10. The computer-readable storage medium of claim 9, wherein the sensor is to verify whether the first item was used in the first healthcare procedure for the first patient.

11. The computer-readable storage medium of claim 10, wherein, when the first item was used in the first healthcare procedure for the first patient, a preference card is updated based on the first item.

12. The computer-readable storage medium of claim 9, wherein the sensor is incorporated into at least one of glasses or an eye shield, and wherein information regarding the first item is displayed via the at least one of glasses or eye shield.

13. The computer-readable storage medium of claim 9, wherein the sensor is incorporated into a cart with a computing device.

14. The computer-readable storage medium of claim 8, wherein the digital twin is periodically retrained and redeployed based on feedback including at least one of the update or the log.

15. A method comprising:
receiving, using a processor, input regarding a first item at a first location;
comparing, using the processor, the first item to items associated with each task of a first healthcare procedure, the items associated with each task of the first healthcare protocol modeled using a digital twin of the first healthcare protocol, the digital twin including a data structure created from tasks defining the first healthcare procedure and a list of items to be used in the first healthcare procedure to model the tasks of the first healthcare procedure and items associated with each task of the first healthcare procedure, the digital twin arranged for query and simulation via the processor to model the first healthcare procedure for a first patient;
when the first item matches an item associated with a task of the first healthcare procedure, recording the first item and approval for the first healthcare procedure and update the digital twin based on the first item; and
when the first item does not match an item associated with a task of the first healthcare procedure, logging the first item.

16. The method of claim 15, wherein the digital twin is to interact with a sensor to identify the first item at the first location.

17. The method of claim 16, wherein the sensor is to verify whether the first item was used in the first healthcare procedure for the first patient.

18. The method of claim 17, wherein, when the first item was used in the first healthcare procedure for the first patient, the method further includes updating a preference card based on the first item.

19. The method of claim 15, wherein the sensor is incorporated into at least one of glasses, an eye shield, or a cart, and wherein information regarding the first item is displayed via the at least one of glasses, eye shield, or cart.

20. The method of claim 15, further including periodically retraining and redeploying the digital twin based on feedback including at least one of the updating or the logging.
